(19) United States
(12) Patent Application Publication
(10) Pub. No.: US 2024/0102981 A1
(43) Pub. Date: Mar. 28, 2024

(54) METHOD FOR AUGMENTING DATASETS

(71) Applicant: Aclima Inc., San Francisco, CA (US)

(72) Inventor: Ricardo T. Lemos, San Francisco, CA (US)

(21) Appl. No.: 18/368,944

(22) Filed: Sep. 15, 2023

Related U.S. Application Data
(60) Provisional application No. 63/407,536, filed on Sep. 16, 2022.

(51) Int. Cl. G01N 33/00

(52) U.S. Cl. CPC G01N 33/0062

(57) ABSTRACT

A system, device, and method for augmenting environmental data is disclosed. The method includes (i) obtaining an environmental dataset having a first data resolution, and (ii) determining an augmented environmental dataset based at least in part on the environmental dataset, a set of spatial features, a set of temporal features, and a set of spatiotemporal features. The model has a second data resolution that is finer than the first data resolution.

### CROSS REFERENCE TO OTHER APPLICATIONS

This application claims priority to U.S. Provisional Patent Application No. 63/407,536 entitled METHOD FOR AUGMENTING ENVIRONMENTAL DATA RESOLUTION filed Sep. 16, 2022 which is incorporated herein by reference for all purposes.

### BACKGROUND OF THE INVENTION

Monitoring of environmental conditions includes measuring the levels of various components of the surroundings, allowing detection of potentially harmful air pollution, radiation, greenhouse gases, or other contaminants in the environment. Monitoring of environmental conditions typically includes gathering environmental data. Environmental data includes detection and measurement of pollutants or contaminants such as nitrogen dioxide (NO₂), carbon monoxide (CO), nitrogen oxide (NO), ozone (O₃), sulfur dioxide (SO₂), carbon dioxide (CO₂), methane (CH₄), volatile organic compounds (VOC), air toxics, temperature, sound radiation, and particulate matter. In order to assess the effects of such pollutants, it is desirable to associate environmental data sensing these pollutants at particular times with geographic locations (homes, businesses, towns, etc.). Such an association would allow individuals and communities to evaluate the quality of their surroundings.

Environmental data collected over time period and geographic region is generally sparsely populated along a spatial dimension and/or temporal dimension. Plausible values for environmental data at locations or times for which an observation is not collected may be predicted to populate an environmental dataset. Thus, a mechanism for improving collection and processing of environmental data is desired.

### BRIEF DESCRIPTION OF THE DRAWINGS

Various embodiments of the invention are disclosed in the following detailed description and the accompanying drawings.

FIG. 1 illustrates an embodiment of a system for capturing environmental data using mobile sensor platforms and associating the environmental data with map features.

FIG. 2 illustrates an embodiment of a method for capturing environmental data using mobile sensor platforms.

FIGS. 3A-3C illustrate a particular region and the embodiment of routes that may be traversed using a method for capturing environmental data using mobile sensor platforms.

FIGS. 4A-4D illustrate examples of a pre-processing performed with respect to collected environmental data according to various embodiments.

FIG. 5A illustrates an example of an environmental data baseline generated by a raw mean of pass values according to various embodiments.

FIG. 5B illustrates an example of an environmental data baseline generated by a spatial module according to various embodiments.

FIG. 6A illustrates an example of a model of the effect of elevation on pollutant concentrations according to various embodiments.

FIG. 6B illustrates an example of a model of the effect or a road type on pollutant concentrations according to various embodiments.

FIGS. 7A and 7B illustrate examples of histograms of null model and spatial module residuals for a particular pollutant according to various embodiments.

FIG. 8 illustrates an example of temporal dynamics of spatially averaged residuals from a spatial module for a particular pollutant and predictions from a weather module according to various embodiments.

FIG. 9 illustrates a decomposition of an environmental dataset to contributions from the effects of a set of features according to various embodiments.

FIG. 10 illustrates a process for analyzing an environmental dataset to determine contributions from the effects of a set of features according to various embodiments.

FIG. 11 illustrates a process for using a DINEOF/Kalman Filter (DKF) filter to analyze an environmental dataset to determine contributions from the effects of a set of features according to various embodiments.

FIG. 12 illustrates a process for using a bias normalization to analyze an environmental dataset to determine contributions from the effects of a set of features according to various embodiments.

FIG. 13A illustrates a spatial component for a pollutant concentration according to various embodiments.

FIG. 13B illustrates a baseline for a pollutant concentration over a geographic region according to various embodiments.

FIG. 14 illustrates the components for weather, diurnal cycle, and nonlinear trends for an environmental dataset according to various embodiments.

FIG. 15A illustrates a hyperlocal variability component of an environmental dataset for a particular pollutant extracted using a DKF filter.

FIGS. 15B-15D illustrate examples of principal component (PC) loadings obtained from an environmental dataset for a particular pollutant extracted using a DKF filter.

FIG. 16 illustrates an example of the various contributions, from a set of features to an environmental dataset for various pollutants.

FIG. 17A illustrates an example of a variance for contributions associated with a set of features for various pollutants.

FIG. 17B illustrates an example of the root-mean-square error (RMSE) associated with a model for a set of contracts across various pollutants.

FIG. 18A illustrates a comparison of a DKF model and a residual model for air quality (REMAQ) model generated for an environmental dataset corresponding to a particular pollutant according to various embodiments.

FIG. 18B illustrates a comparison of a DKF model and a residual model for air quality (REMAQ) model generated for an environmental dataset corresponding to a particular pollutant according to various embodiments.

FIG. 19 illustrates a method for augmenting environmental data according to various embodiments.

FIG. 20 illustrates a method for pre-processing environmental data according to various embodiments.

FIG. 21 illustrates a method for determining the components for a set of features that are contributed to a prediction of environmental data according to various embodiments.

FIG. 22 illustrates a method for determining the components for a set of features that are contributed to a prediction of environmental data according to various embodiments.

FIG. 23 illustrates a method for determining an augmented environmental dataset according to various embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

As used herein, road type includes a classification of a particular road segment. The classification may be determined by a third-party map service. For example, open street maps (OSM) comprises labels attributed to different road segments. Examples of road types include highway, major roads, residential roads, etc. Additional information for each road may indicate a number of lanes, a road surface, a maximum speed, a minimum speed, an indication that the corresponding road segment is within a school zone, an indication that the corresponding road segment is within a construction zone, etc.

Generally, environmental data cannot be sampled everywhere in a predefined geographic region (e.g., a contract region over which environmental data, such as pollutant concentrations are to be measured) at all times continuously. The system can use the environmental data to generate maps or other representations or time series, or to make predictions of environmental characteristics (e.g., pollutant concentrations) at a particular location and a particular point in time. Effective use of the environmental data to represent or predict an environmental characteristic generally necessitates the system to ensure that the quality of environmental data used to generate the representations or prediction is accurate. As such, the system generally takes into consideration invalid or inaccurate sampling.

According to various embodiments, the system augments an environmental dataset with best guesses. The environmental dataset may be a dataset of collected samples such as sampling obtained during a session in which a vehicle travels according to a predefined drive plan. Augmenting the environmental dataset with best guesses includes imputing values for environmental data at locations and/or times that are unsampled. The system may use the sampled environmental data and one or more models (e.g., statistical models) of the effects of different features (e.g., spatial features such as topography or road type, temporal features, and/or spatiotemporal features). In some embodiments, the system determines the one or more models based on an environmental dataset (e.g., data collected over a predefined period of time, such as a contract length for pollutant monitoring) to determine the contributions made to the collected environmental data by one or more features to predict the collected environmental data. The system obtains the environmental dataset, extracts information from the environmental data comprised in the environmental dataset (e.g., spatial or temporal structures), and imputes observations to a particular location for a particular time.

Various embodiments provide a system, method, and device for augmenting environmental data. The method includes (i) obtaining an environmental dataset having a first data resolution, and (ii) determining an augmented environmental dataset based at least in part on the environmental dataset, a set of spatial features, a set of temporal features, and a set of spatiotemporal features. The model has a second data resolution that is finer than the first data resolution. In some embodiments, the model has less null values for a cell corresponding to a predefined location at a predefined time than the environmental dataset. In some embodiments, the model has no missing values.

Various embodiments provide a system, method, and device for augmenting environmental data. The method includes (i) obtaining a sparse environmental dataset, and (ii) determining an augmented environmental dataset based at least in part on the environmental dataset, a set of spatial features, a set of temporal features, and a set of spatiotemporal features. The resulting augmented environmental dataset has no missing values.

Various embodiments provide a system, method, and device for augmenting environmental data. The method includes (i) obtaining an environmental dataset (e.g., a sparse environmental dataset), and (ii) determining an augmented environmental dataset based at least in part on the environmental dataset, a set of spatial features, a set of temporal features, and a set of spatiotemporal features. The augmented environmental dataset being less sparse than the environmental dataset. In some embodiments, the augmented environmental dataset has less missing or null values than a the environmental dataset. In some embodiments, a system uses a Data Interpolating Empirical Orthogonal Functions (DINEOF)/Kalman Filter (KF) (DKF) to determine imputed values for a sparsely populated environmental dataset. The system uses the DKF to leverage the spatiotemporal correlations between measurements (e.g., sampled observations) to perform imputation of values to populate the environmental dataset (e.g., in connection with determining an augmented environmental dataset). The system uses a DKF on an environmental dataset having a particular temporal resolution (e.g., a daily temporal resolution) to impute values to empty cells in a matrix representation of the environmental dataset. In some embodiments, the system imputes the values without using any extraneous or information outside of the collected environmental data. For example, the system imputes the values based on the particular pollutant being analyzed. As an illustrative example, if the system is monitoring/analyzing ozone, then the system generates an augmented dataset that comprises the collected ozone dataset combined with the associated imputed values that are generated based on the ozone dataset without other information.

In some embodiments, the system pre-processes the environmental dataset (e.g., pass data from a set of driving sessions) to obtain a decycled residuals data by removing road-type-specific diurnal cycles (e.g., contributions from morning and evening rush hours). Diurnal cycles may also arise based on reactions with pollutants that are dependent on radiation (e.g., ozone, nitrogen dioxide, etc.), photochemistry effects on the pollutants, solar radiation that may drive or contribute to production cycles, or other loss mechanisms (e.g., humidity, time of day, etc.). In some embodiments, the system uses DKF to obtain a dataset of daily environmental characteristic measurements (e.g., daily pollutant concentrations), for all road segments within a predefined geographic region (e.g., a contracted region). To obtain the dataset of daily environmental characteristic measurements the system uses the decycled residuals data. As an example, the system removes the diurnal cycle from the raw pass data, so that the resulting hyperlocal daily dataset would not be full of spurious bumps and troughs (e.g., nearby roads might appear to have very different levels of pollution, simply because such roads were sampled at different times of day). To remove the cycle, the system subtracts a road-type-specific estimate of the diurnal cycle from each observation. The system can determine the noise contribution to the environmental data based on the identified hyperlocal variability.

Various embodiments build on the use of DKF by using a residual model for air quality (REMAQ) to perform imputation. The REMAQ includes the use of regression analysis (e.g., a generalized additive model) in conjunction with a DKF to obtain imputed environmental data.

In some embodiments, the system implements a residual model for air quality (REMAQ) in conjunction with a DKF to impute values to an environmental dataset in connection with imputing values to an augmented environmental dataset or otherwise making a prediction. The system uses the REMAQ to identify the contributions made by a set of features to the environmental dataset. In some embodiments, the system uses information outside the collected environmental dataset to determine the feature-specific components in the environmental dataset. For example, the system uses one or more of topography (e.g., latitude, longitude, altitude), weather, road type, and other features (e.g., correlations associated with other pollutants at the particular location and time) to make predictions of environmental data at particular locations and particular times. In some embodiments, the use of the REMAQ enables better and higher resolution predictions for imputed values.

Various features outside the collected environmental data may be used in connection with determining an augmented environmental dataset or otherwise predicting environmental data for a particular location and time. Examples of such features include other pollutants (e.g., data from other pollutants that may have a causal relationship with the pollutant being analyzed), weather, topography, road characteristics, etc. Weather includes one or more of temperature, precipitation level, visibility, wind speed (e.g., decomposing a wind vector into a zonal wind speed component and/or a meridional wind speed component), downwards radiation, height of the planetary boundary layer, etc. Topography may include one or more of latitude, longitude, and altitude. Examples of road characteristics include road type, road width, road surface, number of lanes, traffic information (e.g., historical traffic and/or current traffic), road construction zones, etc.

The system uses regression analysis to incorporate extraneous environmental predictors (e.g., features other than the collected measurements). An example of a type of environmental monitoring includes the monitoring/modeling of pollution at a particular point and time. In some embodiments, the system deems pollution (e.g., pollutant concentrations) to be a function of a plurality of different variables (e.g., features), such as road type, altitude or the road segment, weather (e.g., weather occurring when a sample is collected, or weather occurring when a prediction is being generated), etc. The system uses regression to estimate the coefficients for the variables. As an example, the system uses a generalized additive model in connection with determining the contributions to the predictions of environmental data made by the various features (e.g., a set of component of the environmental data respectively associated with each of the features). The relationship between the various features and a corresponding pollutant can be non-linear. For example, environmental data is not necessarily a linear combination of the extraneous environmental predictors. As an illustrative example, an increase in altitude, from 0 to 100 m, does not entail the same change in pollutant concentration as a change in altitude from 100 to 200 m.

In some embodiments, the system pre-processes the environmental data (e.g., sampled environmental data within the environmental dataset) before using the environmental data to generate models (e.g., determine a contribution by a particular feature to the prediction of environmental data) or make predictions (e.g., determine imputed values, etc.). Pre-processing the data enables the system to use high-quality data to build the models and make predictions. Pre-processing the environmental data includes updating the environmental data to adjust/remove non-sensical data, etc. For example, the pre-processing includes adjusting the environmental data to resolve negative concentrations. In some embodiments, when selecting which features to use in predicting environmental data (e.g., determining the imputed values and/or augmented environmental dataset), the system naturally prefers those features that have plausible causal nature, but sometimes the system may be required to use proxies (e.g., number of lanes, maximum speed, etc.) as a surrogate for unavailable features that the system would much rather have (e.g. emissions from ICE vehicles).

In response to determining the models, the system may validate the models before deployment. In various embodiments, the system validates the models based at least in part on one or more of (a) assessing an internal goodness-of-fit (e.g., qq plots, histograms, correlograms, etc.); (b) determine whether the Kalman filter (KF) initial conditions are important, such as by examining initial time points; (c) perform out-of-sample validation, such as assessing a root mean square error (RMSE), (d) validate against synthetic data; and (e) validate against third party data (e.g., regulatory stations, purpleAir, etc.).

Hyper-local environmental data, for example related to air quality and greenhouse gas data, can be collected using vehicles with air pollutant sensors installed. Embodiments of techniques usable in gathering hyper-local data are described in U.S. patent application Ser. No. 16/682,871, filed on Nov. 13, 2019, entitled HYPER-LOCAL MAPPING OF ENVIRONMENTAL CONDITIONS and assigned to the assignee of the present application, U.S. patent application Ser. No. 16/409,624, filed on May 10, 2019, entitled INTEGRATION AND ACTIVE FLOW CONTROL FOR ENVIRONMENTAL SENSORS and assigned to the assignee of the present application; U.S. patent application Ser. No. 16/773,873, filed on Jan. 27, 2020, entitled SENSOR DATA AND PLATFORMS FOR VEHICLE ENVIRONMENTAL QUALITY MANAGEMENT, assigned to the assignee of the present application and which claims priority to U.S. Patent Application Ser. No. 62/798,395 entitled SENSOR DATA AND PLATFORMS FOR VEHICLE ENVIRONMENTAL QUALITY MANAGEMENT and assigned to the assignee of the present application, which are all incorporated herein in their entirety for all purposes.

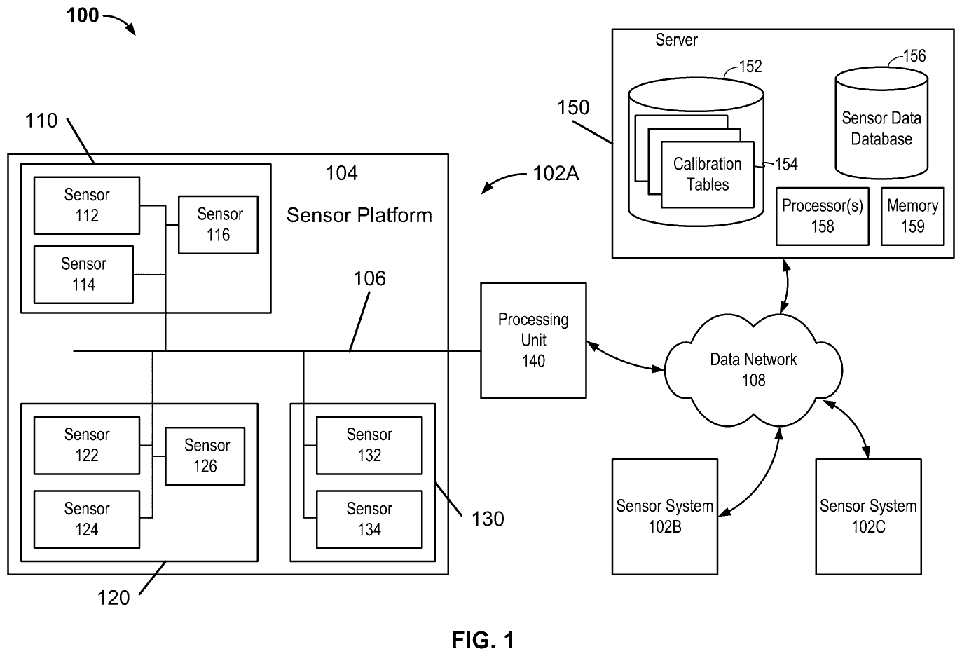

FIG. 1 depicts an embodiment of a system 100 for collecting and processing environmental data. System 100 includes multiple mobile sensor platforms 102A, 102B, 102C and server 150. In some embodiments, system 100 may also include one or more stationary sensor platforms 103, of which one is shown. Stationary sensor platform 103 may be used to collect environmental data at a fixed location. The environmental data collected by stationary sensor platform 103 may supplement the data collected by mobile sensor platforms 102A, 102B and 102C. Thus, stationary sensor platform 103 may have sensors that are the same as or analogous to the sensors for mobile sensor platforms 102A, 102B and 102C. In other embodiments, stationary sensor platform 103 may be omitted. Although a single server 150 is shown, multiple servers may be used. The multiple servers may be in different locations. Although three mobile sensor platforms 102A, 102B and 102C are shown, other numbers of sensors/mobile sensor platforms are typically present. Mobile sensor platforms 102A, 102B and 102C and stationary sensor platform(s) 103 may communicate with server 150 via a data network 108. The communication may take place wirelessly.

Mobile sensor platforms 102A, 102B and 102C may be mounted in a vehicle, such as an automobile or a drone. In some embodiments, mobile sensor platforms 102A, 102B and 102C are desired to stay in proximity to the ground to be better able to sense conditions analogous to what a human would experience. Mobile sensor platform 102A includes a bus 106, sensors 110, 120 and 130. Although three sensors are shown, another number may be present on mobile sensor platform 102A. In addition, a different configuration of components may be used with sensors 110, 120 and 130. Each sensor 110, 120 and 130 is used to sense environmental quality and may be of primary interest to a user of system 100. For example, sensors 110, 120 and 130 may be gas sensors, volatile organic compound (VOC) sensors, particulate matter sensors, radiation sensors, noise sensors, light sensors, temperature sensors, noise sensors or other analogous sensors that capture variations in the environment. For example, sensors 110, 120 and 130 may be used to sense one or more of NO₂, CO, NO, O₃, SO₂, CO₂, VOCs, CH₄, particulate matter, noise, light, temperature, radiation, and other compounds. In some embodiments, sensor 110, 120 and/or 130 may be a multi-modality sensor. A multi-modality gas sensor senses multiple gases or compounds. For example, if sensor 110 is a multi-modality NO₂/O₃ sensor, sensor 110 might sense both NO₂ and O₃ together. Sensor 110 may comprise a plurality of sensors, such as sensors 112, 114, and 116. Sensor 120 may comprise a plurality of sensors, such as sensors 122, 124, and 126. Sensor 130 may comprise a plurality of sensors, such as sensors 132 and 134.

Although not shown in FIG. 1, other sensors co-located with sensors 110, 120 and 130 may be used to sense characteristics of the surrounding environment including, in some instances, other gases and/or matter. Such additional sensors are exposed to the same environment as sensors 110, 120 and 130. In some embodiments, such additional sensors are in close proximity to sensors 110, 120 and 130, for example within ten millimeters or less. In some embodiments, the additional sensors may be further from sensors 110, 120 and 130 if the additional sensors sample the same packet of air inside of a closed system, such as a system of closed tubes. In some embodiments, temperature and/or pressure are sensed by these additional sensors. For example, an additional sensor co-located with sensor 110 may be a temperature, pressure, and relative humidity (T/P/RH) sensor. These additional co-located sensors may be used to calibrate sensors 110, 120 and/or 130. Although not shown, sensor platform 102A may also include a manifold for drawing in air and transporting air to sensors 110, 120 and 130 for testing.

Sensors 110, 120 and 130 provide sensor data over bus 106, or via another mechanism. In some embodiments, data from sensors 110, 120 and 130 incorporates time. This time may be provided by a master clock (not shown) and may take the form of a timestamp. Master clock may reside on sensor platform 102A, may be part of processing unit 140, or may be provided from server 150. As a result, sensors 110, 120 and 130 may provide timestamped sensor data to server 150. In other embodiments, the time associated with the sensor data may be provided in another manner. Because sensors 110, 120 and 130 generally capture data at a particular frequency, sensor data is discussed as being associated with a particular time interval (e.g., the period associated with the frequency), though the sensor data may be timestamped with a particular value. For example, sensors 110, 120 and/or 130 may capture sensor data every second, every two seconds, every ten seconds, or every thirty seconds. The time interval may be one second, two seconds, ten seconds, or thirty seconds. The time interval may be the same for all sensors 110, 120 and 130 or may differ for different sensors 110, 120 and 130. In some embodiments, the time interval for a sensor data point is centered on the timestamp. For example, if the time interval is one second and a timestamp is t1, then the time interval may be from t1−0.5 seconds to t1+0.5 seconds. However, other mechanisms for defining the time interval may be used.

Sensor platform 102A also includes a position unit 145 that provides position data. In some embodiments, position unit 145 is a global positioning satellite (GPS) unit. Consequently, system 100 is described in the context of a position unit 145. The position data may be time-stamped in a manner analogous to sensor data. Because position data is to be associated with sensor data, the position data may also be considered associated with time intervals, as described above. However, in some embodiments, position data (e.g., GPS data) may be captured more or less frequently than sensor data. For example, position unit 145 may capture position data every second, while sensor 130 may capture data every thirty seconds. Thus, multiple data points for the position data may be associated with a single thirty second time interval. The position data may be processed as described below.

Optional processing unit 140 may perform some processing and functions for data from sensor platform 104, may simply pass data from sensor platform 104 to server 150 or may be omitted.

Mobile sensors platforms 102B and 102C are analogous to mobile sensor platform 102A. In some embodiments, mobile sensor platforms 102B and 102C have the same components as mobile sensor platform 102A. However, in other embodiments, the components may differ. However, mobile sensor platforms 102A, 102B and 102C function in an analogous manner.

Server 150 includes sensor data database 156, calibration tables 154 (e.g., stored in database 152), processor(s) 158, memory 159. Processor(s) 158 may include multiple cores. Processor(s) 158 may include one or more central processing units (CPUs), one or more graphical processing units (GPUs) and/or one or more other processing units. Memory 159 can include a first primary storage, typically a random-access memory (RAM), and a second primary storage area, typically a non-volatile storage such as solid-state drive (SSD) or hard disk drive (HDD). Memory 159 stores programming instructions and data for processes operating on processor(s) 158. Primary storage typically includes basic operating instructions, program code, data and objects used by processor(s) 158 to perform their functions. Primary storage devices (e.g., memory 159) may include any suitable computer-readable storage media, described below, depending on whether, for example, data access needs to be bi-directional or uni-directional.

Sensor data database 156 includes data received from mobile sensor platforms 102A, 102B and/or 102C. After capture by mobile sensor platform 102A, 102B and/or 102C, sensor data stored in sensor data database 156 may be operated on by various analytics, as described below. Position data database 152 stores position data received from mobile sensor platforms 102A, 102B and/or 102C. In some embodiments, sensor data database 156 stores position data as well as sensor data. In such embodiments, position data database 152 may be omitted. Server 150 may include other databases and/or store and utilize other data. For example, server 150 may include calibration data (not shown) used in calibrating sensors 110, 120 and 130.

System 100 may be used to capture, analyze, and provide information regarding hyper-local environmental data. Mobile sensor platforms 102A, 102B and 102C may be used to traverse routes and provide sensor and position data to server 150. Server 150 may process the sensor data and position data. Server 150 may also assign the sensor data to map features corresponding to the locations of mobile sensor platforms 102A, 102B and 102C within the same time interval as the sensor data was captured. As discussed above, these map features may be hyper-local (e.g., one hundred meter or less road segments or thirty meter or less road segments). Thus, mobile sensor platforms 102A, 102B and 102C may provide sensor data that can capture variations on this hyper-local distance scale. Server 150 may provide the environmental data, a score, confidence score and/or other assessment of the environmental data to a user. Thus, using system 100 hyper-local environmental data may be obtained using a relatively sparse network of mobile sensor platforms 102A, 102B and 102C, associated with hyper-local map features and processed for improved understanding of users.

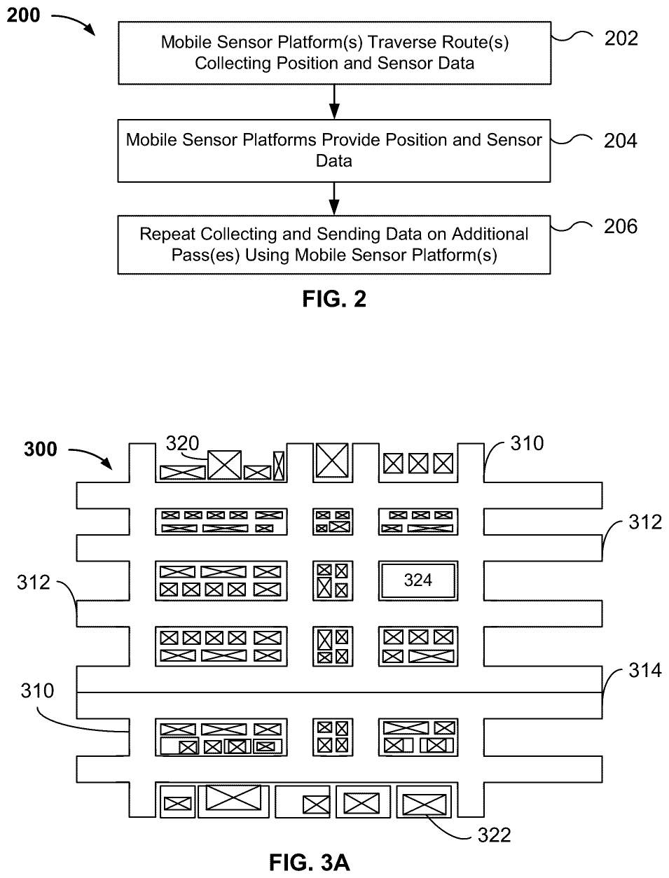

FIG. 2 depicts an exemplary embodiment of method 200 for capturing environmental data using mobile sensor platforms, such as mobile sensor platforms 102A, 102B and/or 102C. Method 200 is described in the context of system 100, but may be performed using other systems. For clarity, only some portions of method 200 are shown. Although shown in a sequence, in some embodiments, processes may occur in parallel and/or in a different order.

Mobile sensor platforms traverse routes in a geographic region, at 202. While traversing the routes, the mobile sensor platforms collect not only sensor data, but also position data. For example, a mobile sensor platform may sense one or more of NO₂, CO, NO, O₃, SO₂, CO₂, CH₄, VOCs, particulate matter, other compounds, radiation, noise, light, and other environmental data at various times during traversal of the route. Other environmental characteristics, including but not limited to temperature, pressure, and/or humidity may also be sensed at 202. In addition, the time corresponding to the environmental data is also captured. The time may be in the form of a timestamp for the sensor data (sensor timestamp), which may correspond to a particular time interval. Different sensors on the mobile sensor platform may capture the environmental data at different times and/or at different frequencies. Also, at 202 the mobile sensor platforms capture position data, for example via a GPS unit. The position data may include location (as indicated by a GPS unit), velocity and/or other information related to the geographic location of the mobile sensor platform. In some embodiments, position data from other sources, such as acceleration, may be captured from by the vehicle or another source. The position data may include a timestamp (position timestamp) or other indicator of the time at which the position data is captured.

The mobile sensor platforms provide the position and sensor data to a server, at 204. In some embodiments, mobile sensor platforms provide this data substantially in real time, as the mobile sensor platforms traverse their routes at 202. Thus, the position and sensor data may be transmitted wirelessly to the server. In some embodiments, some or all of the position and/or sensor data is stored at the mobile sensor platform and provided to the server at a later time. For example, the data may be transferred to the server when the mobile sensor platform returns to its base. In some embodiments, the mobile sensor platform may process the sensor data and/or position data prior to sending the sensor and/or position data to the server. In other embodiments, the mobile sensor platform provides little or no processing. The sensor data and position data may be sent at the same time or may be sent separately.

At 206, the route traversal and data collecting of 202 and data sending of 204 are repeated. Thus, the mobile sensor platforms may traverse the same or different routes at 206. In either case, multiple passes of the same geographic locations, and thus multiple passes of the same corresponding map features, are made at 206. In some embodiments, the repetition at 206 may be periodic (e.g., approximately every week, month, or other time period). In some embodiments, the repetition at 206 may be performed based on other timing. In some cases, the same mobile sensor platform is sent on the same route and/or collects data for the same map features. In some embodiments, different mobile sensor platforms collect data may be used for the same routes and/or map features. Also at 206, steps 202 and 204 may be performed multiple times. Thus, at 206, data for a particular region may be aggregated over time.

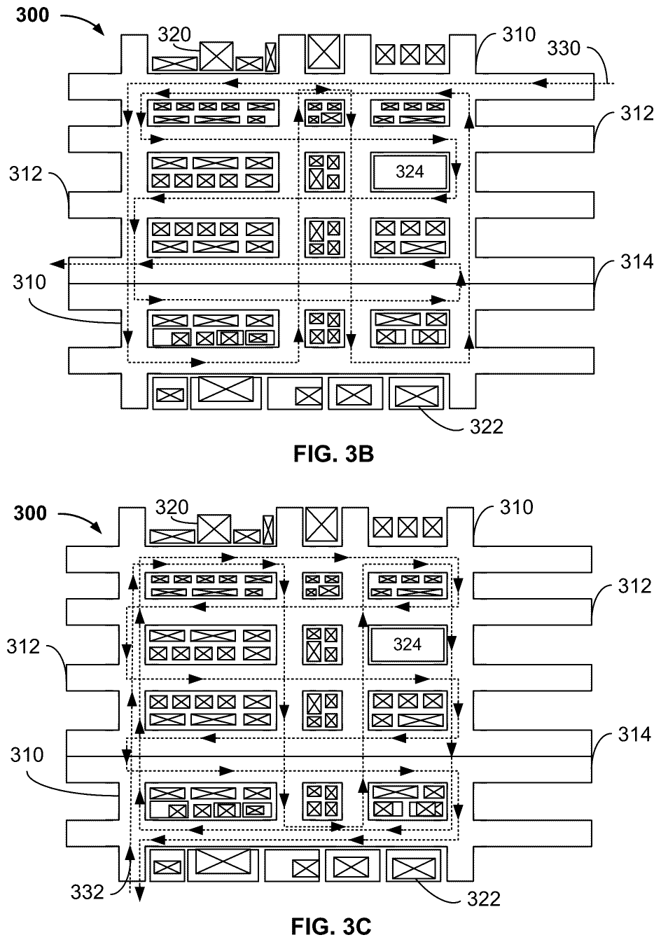

For example, FIGS. 3A-3C illustrate a particular geographic region and the routes that may be traversed using method 200. A map 300 corresponding to the geographic region is shown in FIG. 3A. Map 300 may be an open-source map or generated by another mapping tool. Map 300 includes streets 310 (oriented vertically on the page) and 312 (oriented horizontally on the page); larger street/highway 314, structures 320 and 322 and open area 324. For simplicity, only one of each structure 320 and 322 is labeled. Open area 324 may correspond to a park, vacant lot, or analogous item. As can be seen in FIG. 3A, the density and size of structures 320 and 322 vary across map 300. Similarly, the density and size of streets 312, 314 and 320 also varies. In addition, structures 322 are more clearly separated by open regions, which may correspond to a yard or analogous area.

FIG. 3B illustrates map 300 as well as route 330 that may be traversed by a mobile sensor platform, such as mobile sensor platform 102A. At 202, mobile sensor platform 102A may traverse route 330. As can be seen in FIG. 3B, the route 330 includes a portion of each street 312 and 314 in map 300. Some portions of some streets are traversed multiple times for the same route 330. In some embodiments, this is still considered a single pass of these streets. As mobile sensor platform 102A traverses route 330 at 202, sensor data is captured by sensors 110, 120 and 130. Also at 202, position data is captured by position unit 145 throughout route 330. In some embodiments, the vehicle carrying mobile sensor platform 102A travels sufficiently slowly while traversing route 330 that sensor data and position data can be accurately captured for particular position(s). In some embodiments, mobile sensor platform 102A travels at a velocity that allows for multiple sensor data points for each map feature. Mobile sensor platform 102A also sends position and sensor data to server 150 at 204. This may be done while mobile sensor platform 102A traverses route 330 or at a later time. Other mobile sensor platforms 102B and/or 102C may also traverse the same or different routes and send data to server 150 at 202 and 204. Thus, multiple mobile sensor platforms may be used in method 200.

At 206, mobile sensor platform 102A and/or other mobile sensor platform(s) 102B and 102C repeat the route traversal, data collection and sending of the position and sensor data. In some cases, mobile sensor platform(s) 102A, 102B and/or 102C follow route 330 again. In some cases, mobile sensor platform(s) 102A, 102B and/or 102C traverses a different route. For example, FIG. 3C depicts map 300 with another route 332. As part of 206, mobile sensor platform(s) 102A, 102B and/or 102C may traverse route 332, collecting position and sensor data at 206 (repeating 202). In some embodiments, the vehicle carrying mobile sensor platform(s) 102A, 102B and/or 102C travels sufficiently slowly while traversing route 332 that sensor data and position data can be accurately captured for particular position(s). In some embodiments, mobile sensor platform(s) 102A, 102B and/or 102C travels at a velocity that allows for multiple sensor data points for each map feature (described below). Mobile sensor platform(s) 102A, 102B and/or 102C send sensor and position data to server 150 at 206 (repeating 204) during or after traversing route 330 and/or route 332.

Thus, using method 200, sensor and position data may be captured for regions of a map. The sensor data and position data may be provided to server 150 or other component for processing, aggregation, and analysis. Sensor data and position data are sensed sufficiently frequently using method 200 that variations environmental quality on the hyper-local scales may be reflected in the sensor data. Method 200 may be performed using a relatively small number of mobile sensor platforms. Consequently, efficiency of data gathering may be improved while maintaining sufficient sensitivity in both sensor and position data.

For example, FIGS. 3A-3C illustrate a particular geographic region and the routes that may be traversed using method 200. A map 300 corresponding to the geographic region is shown in FIG. 3A. Map 300 may be an open-source map or generated by another mapping tool. Map 300 includes streets 310 (oriented vertically on the page) and 312 (oriented horizontally on the page); larger street/highway 314, structures 320 and 322 and open area 324. For simplicity, only one of each structure 320 and 322 is labeled. Open area 324 may correspond to a park, vacant lot, or analogous item. As can be seen in FIG. 3A, the density and size of structures 320 and 322 vary across map 300. Similarly, the density and size of streets 312, 314 and 320 also varies. In addition, structures 322 are more clearly separated by open regions, which may correspond to a yard or analogous area.

FIG. 3B illustrates map 300 as well as route 330 that may be traversed by a mobile sensor platform, such as mobile sensor platform 102A. At 202, mobile sensor platform 102A may traverse route 330. As can be seen in FIG. 3B, the route 330 includes a portion of each street 312 and 314 in map 300. Some portions of some streets are traversed multiple times for the same route 330. In some embodiments, this is still considered a single pass of these streets. As mobile sensor platform 102A traverses route 330 at 202, sensor data is captured by sensors 110, 120 and 130. Also at 202, position data is captured by position unit 145 throughout route 330. In some embodiments, the vehicle carrying mobile sensor platform 102A travels sufficiently slowly while traversing route 330 that sensor data and position data can be accurately captured for particular position(s). In some embodiments, mobile sensor platform 102A travels at a velocity that allows for multiple sensor data points for each map feature. Mobile sensor platform 102A also sends position and sensor data to server 150 at 204. This may be done while mobile sensor platform 102A traverses route 330 or at a later time. Other mobile sensor platforms 102B and/or 102C may also traverse the same or different routes and send data to server 150 at 202 and 204. Thus, multiple mobile sensor platforms may be used in method 200.

At 206, mobile sensor platform 102A and/or other mobile sensor platform(s) 102B and 102C repeat the route traversal, data collection and sending of the position and sensor data. In some cases, mobile sensor platform(s) 102A, 102B and/or 102C follow route 330 again. In some cases, mobile sensor platform(s) 102A, 102B and/or 102C traverses a different route. For example, FIG. 3C depicts map 300 with another route 332. As part of 206, mobile sensor platform(s) 102A, 102B and/or 102C may traverse route 332, collecting position and sensor data at 206 (repeating 202). In some embodiments, the vehicle carrying mobile sensor platform(s) 102A, 102B and/or 102C travels sufficiently slowly while traversing route 332 that sensor data and position data can be accurately captured for particular position(s). In some embodiments, mobile sensor platform(s) 102A, 102B and/or 102C travels at a velocity that allows for multiple sensor data points for each map feature (described below). Mobile sensor platform(s) 102A, 102B and/or 102C send sensor and position data to server 150 at 206 (repeating 204) during or after traversing route 330 and/or route 332.

Thus, using method 200, sensor and position data may be captured for regions of a map. The sensor data and position data may be provided to server 150 or other component for processing, aggregation, and analysis. Sensor data and position data are sensed sufficiently frequently using method 200 that variations environmental quality on the hyper-local scales may be reflected in the sensor data. Method 200 may be performed using a relatively small number of mobile sensor platforms. Consequently, efficiency of data gathering may be improved while maintaining sufficient sensitivity in both sensor and position data.

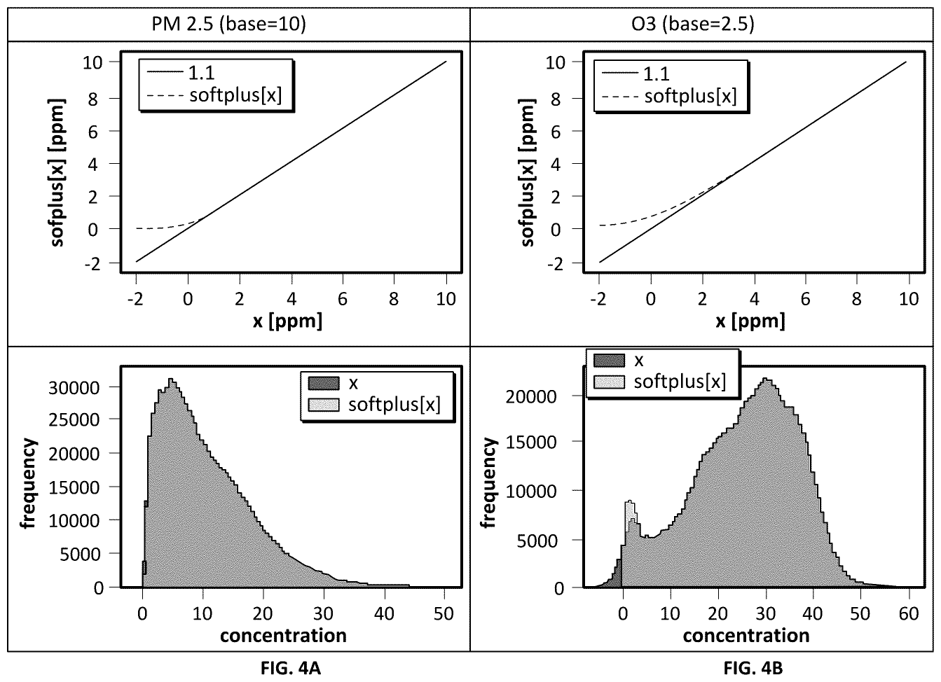

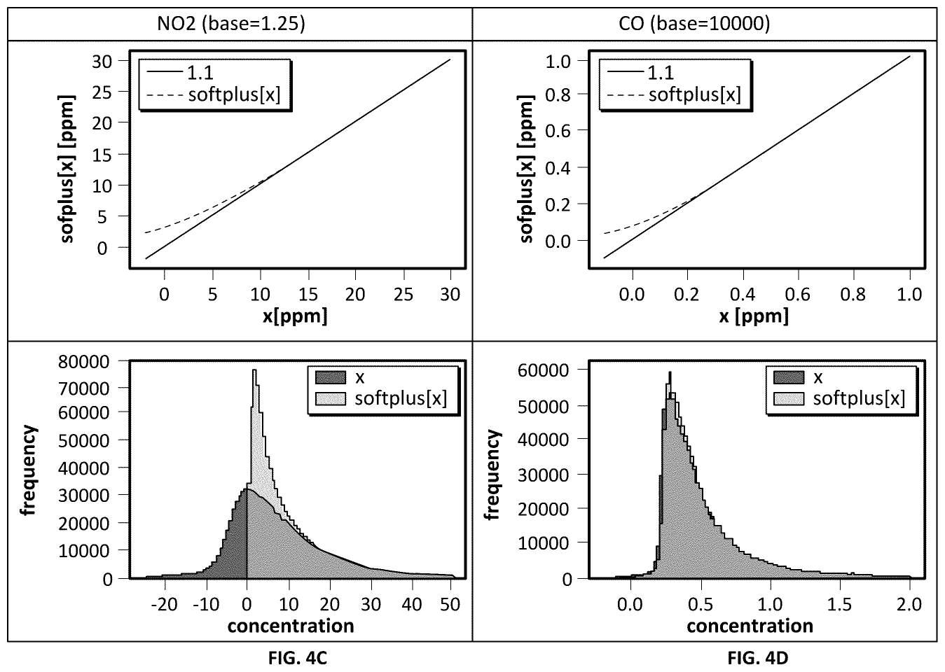

FIGS. 4A-4D illustrate examples of a pre-processing performed with respect to collected environmental data according to various embodiments. In the examples shown, the system adjusts the sampled environmental data to resolve negative concentrations for the various pollutants PM 2.5 (fine particulates), ozone, nitrogen dioxide, and carbon monoxide.

The system uses REMAQ based on collected environmental data (e.g., raw pass data) as input. Generally, each measurement includes an average of 1 Hz measurements taken by a mobile sensor as the mobile sensor travelled a road segment (e.g., a stretch of road roughly 100 meters long). For each session, the system may generate (e.g., probabilistically) a drive plan to provide good spatial diversity and temporal diversity in the environmental dataset (e.g., a set of collected samples over a set of sessions). The number of 1 Hz measurements that go into a pass average depends not only on segment length but also the sensor's speed over ground. For some pollutants, the average (or minimum) distance between the sensor and other vehicles might also affect the final pass average. In some embodiments, the system accounts for these factors. In other embodiments, the system does not take such factors into consideration and takes the pass “measurement” at face value.

The sampled environmental data may comprise noise (e.g., a noise component or contribution to the prediction of environmental data). For example, the mobile sensor deployed to collect the sampled environmental data may introduce noise or measurement errors. These noise/measurement errors generally do not cancel out at the pass level. As shown in FIG. 4C, nitrogen dioxide (NO₂) sampled environmental data (e.g., pass measurements), for example, are frequently negative (e.g., up to roughly 50%) over the course of collecting environmental data for a geographic region over a predetermined period of time (e.g., a contract length, such as a year, a month, etc.). Because pollutant concentrations cannot be negative, the system implements method that corrects these measurements (e.g., to resolve the negative concentrations arising from measurement errors). In some embodiments, the system uses the softplus function, such as the function of Equation 1, to collected environmental data samples (e.g., pass measurements).

$$softplus ( x , κ ) = \frac{1}{log ( κ )} log ( 1 + {κ}^{x} ) \qquad ( 1 )$$

In this expression, the parameter κ>1 controls how early the function begins to approach the 1:1 line. The system determines modality-specific κs based on pass data collected over a predetermined time period (e.g., pass data collected over two years). In some embodiments, the parameter κ is selected to preserve the mode of the original distribution (e.g., the distribution over the raw data samples).

As illustrated in FIGS. 4A-4D, the collected samples corresponding to negative pollutant concentrations are adjusted to be non-negative in a manner that enables the remaining parts of the distribution to closely resemble the original distribution. The system can use the softplus-corrected environmental data as input data to determine the model(s) for predicting environmental data (e.g., imputing values for the environmental data for a particular location at a particular time).

In some embodiments, the system deploys a model(s) to determine contributions (e.g., by a particular feature) to the prediction of environmental data (e.g., spatially-based features, temporally-based features, or spatiotemporally-based features). The model(s) is used in connection with determining predictions for environmental data.

In some embodiments, the system determines the model(s) based on fitting a regression model, such as a generalized additive model, to an environmental dataset. The system uses a generalized additive model to remove the effects of different features (e.g., topography, road type, etc.) and obtains corresponding residual data. The residuals data are obtained because model predictions will generally not match the observed data perfectly. The system can feed the environmental dataset to a generalized additive regression model to determine predictions for all locations in the environmental dataset. For example, the system represents the environmental dataset in a matrix along the dimensions of location and time. The system passes environmental dataset through the additive regression model to impute values to all empty cells in the matrix. The system obtains an augmented environmental dataset from the collected environmental dataset and the imputed values for the empty cells the collected environmental dataset.

In response to obtaining the augmented dataset, the system can remove the predictive contribution of each feature (or a subset of features) from the original environmental dataset (e.g., the dataset of pollutant concentrations), thereby generating a dataset of residuals that are not explained by that feature(s). As an illustrative example, in response to obtaining the augmented dataset, the system determines the effect of topography on the environmental data and removes the topography component to obtain a set of residual data that is not explained by topography, such as data/variability of data that may be explained by other features, such as weather or temporally-based effects.

The system iteratively determines the effect of a particular feature on the environmental data, removes that feature's predicted to contribution to the prediction of environmental data (or input residual data) to obtain residual data that may be used in a subsequent iteration. In some embodiments, at each iteration, the input data (e.g., the environmental dataset on the first pass, or the corresponding residual data for subsequent passes) is fed into a regression analysis to determine the effect of a next feature on the environmental data (e.g., the component for the feature corresponding to the iteration being performed).

In some embodiments, in connection with implementing the REMAQ with an environmental dataset, the system attempts to fit a model to log-transformed, softplus-corrected pass measurements. In other words, the response variable is Yₜ(s)=log(softplus(Yₜ⁰ (s), κ)), where Yₜ⁰ (s) denotes the original pass measurement made at time t and road segment s. The system assumes that the particular random variable follows the Gaussian distribution, with a fairly complex expression for the mean (mₜ(s)) and a simple unknown parameter (σ2) for the variance, as shown in Equation 2.

Yₜ(s)˜N[mₜ(s),σ²] (2)

The mean is provided by Equation 3 below. The constituent parts of Equation 3 are defined by functions such as by the examples described in Equations 4-8.

mₜ(s)=g₁(x^{space}(s))+g₂(xₜ^{weather})+g₃(xₜ^{hour})+g₄(t)+g₅(t,s) (3)

In Equation (3) above:
g₁(x^{space}(s)) corresponds to the nonlinear effect of topography and location on the mean;
g₂(xₜ^{weather}) corresponds to the nonlinear effect of weather on the mean;
g₃(xₜ^{hour}) corresponds to the seasonally-changing diurnal cycle in the mean;
g₄(t) corresponds to the principal component of variability of the mean; and
g₅(t,s) corresponds to hyperlocal variability of the mean.

In the expression for the mean, such as the mean defined by Equation 3, the system utilizes the following underlying assumptions:
the impact of topography and location does not change over time;
the impact of weather does not change across space;
the diurnal cycle does not change across space all road segments share a common, principal component of (temporal) variability; and
violations of the previous four assumptions are mitigated by a term dubbed hyper-local variability, specific to each road segment and time point.

In some embodiments, different models can be determined in which the above-noted assumptions are not made.

The nonlinear functions that connect environmental log-characteristics (e.g., pollutant log-concentrations) to environmental features are defined by Equations 4 and Equation 5 below.

g₁(x^{space}(s))=tensor(latitude(s),longitude(s))+spline(altitude(s))+factor(road type(s)) (4)

g₂(x^{weather}(t))=spline(DLWRFₜ)+spline(HLBLₜ)+spline(PRATEₜ)+spline(TCDCₜ)+spline(TMPₜ)+spline(UGRDₜ)+spline(VGRDₜ) (5)

In Equation 5 above, DLWRF refers to downward longwave radiation flux, HLBL refers to the height of the planetary boundary layer, PRATE refers to the precipitation rate, TCLDC refers to the total cloud cover, TMP refers to the near-surface air temperature, UGRD refers to the near-surface zonal wind speed, and VGRD refers to the near-surface meridional wind speed.

The system accounts for temporal features, such as temporally varying diurnal cycles. As an example, the system implements Equation 6 to account for temporally varying diurnal cycles. Although Equation 6 uses values at an hourly resolution, various other time resolutions may be implemented.

g₂+Spline(1_{hour(t)==12AM})+spline(1_{hour(t)==1AM})+spline(1_{hour(t)==2AM})+ . . . 30 spline(1_{hour(t)==11PM}) (6)

In some embodiments, the system accounts for the principal component of variability, such as based on Equation 7.

g₄(t)=spline(t) (7)

In some embodiments, the system accounts for hyperlocal variability via Equation 8. Hyperlocal variability may correspond to consistent differences in pollutant concentrations between neighboring road segments during a predefined period of time (e.g., a predefined contract). As an example, this equation can be part of the KF.

g₅(t,s)=Σₖ₌₁^{K}ϕ(s)^{[k]}λ_{day(t)}^{[k]} (8)

In Equation 8, K∈N denotes the number of hyperlocal components, φ(s)^{[k]}∈R is the value of the k-th spatial component at location s, and λ_{day(t)}^{[k]} is the k-th coefficient associated with day(t).

The sample size of environmental data in the environmental dataset can be extremely large. The sample size can be sufficiently large that solving for the coefficients for the set of features may be extremely computationally expensive and thus infeasible. In some embodiments, the system solves the problem by solving for different types of features. For example, the system iteratively fits the environmental data to a feature or type of feature.

In some embodiments, the system fits the environmental data by implementing five modules: (1) a spatial module, (2) a weather module, (3) a diurnal cycle module, (4) a principal component module, and (5) a hyperlocal module. The inputs of one module are the residuals of the previous module, hence the name REMAQ. For example, the system begins with the pre-processed environmental data from the environmental dataset and determines the component for a first feature (e.g., a spatial feature), and iteratively determines the component for the remaining features based on residual data from the solution of an immediately preceding feature. Although examples described herein solve the fitting of the environmental dataset to a set of features in the order of spatial effects, temporal effects, spatiotemporal effects, and noise effects, various other orders may be implemented.

The spatial module is used for fitting the data to spatially-based effects to determine the spatial component of (e.g., contribution to the predictions of) the environmental data. In some embodiments, the spatial module employs temporally averaged means as the response variable. An example of the temporally average means is provided in Equation 9.

$$Y ( s ) = \frac{1}{n ( s )} {\sum }_{t} {Y}_{t} ( s ) \qquad ( 9 )$$

The system fits the generalized additive model (GAM) according to Equation 10.

Y(s)=g₁(x^{space}(s))+ϵ(s) (10)

In Equation 10, ϵ(s) denotes spatially uncorrelated Gaussian error. Various embodiments fit this GAM to the environmental data based on Python's library pygam or other similar tools. GAMs provide an easy way to describe the behavior of response variables via smooth, non-linear functions of features.

The weather module is used for fitting the data to weather-based effects to determine the weather component of (e.g., contribution to the prediction of) the environmental data. Based on the predictions from the spatial module (e.g., g₁(x_{space}(s))), the weather module employs spatially-averaged residuals as the response variable. The residuals may be computed from a function such as the function defined in Equation 11.

Yₜ=Σ(Yₜ(s)− [symbol] (x^{space}(s)) (11)

The system fits the GAM according to Equation 12.

Yₜ=g₂(xₜ^{weather})+ϵₜ (12)

In Equation 12, Et denotes temporally uncorrelated Gaussian error.

The diurnal cycle module is used for fitting the data to diurnal cycle-based effects to determine the diurnal cycle component of (e.g., contribution to the prediction of) the environmental data. Based on the predictions from the weather module (e.g., g₂(xₜ^{weather})) the diurnal cycle module employs the resulting residuals computed from a function such as Equation 13 as the response variable.

Yₜ^{†}=Yₜ− [symbol] (xₜ^{weather}) (13)

The system fits the GAM according to Equation 14.

Yₜ^{†}=g₃(xₜ^{hour})+ζₜ (14)

In equation 14, ζₜ denotes temporally uncorrelated Gaussian error.

The principal component module is used for fitting the data to principal component-based effects to determine the contribution of the principal component to the prediction of environmental data. Based on the predictions from the diurnal cycle module (e.g., g₃(xₜ^{hour})), the principal component module employs the resulting residuals computed from a function such as Equation 15.

Yₜ^{‡}=Yₜ^{†}− [symbol] (xₜ^{hour}) (15)

The system fits the GAM according to Equation 16.

Yₜ^{†}=g₄(t)−vₜ (16)

In Equation 16, vₜ denotes temporally uncorrelated Gaussian error.

In some embodiments, the system implements a DKF in connection with determining the hyperlocal-based effects. The system uses a hyperlocal module that fitting the environmental data to hyperlocal-based effects to determine the contribution of the hyperlocal features to the prediction of environmental data.

Data Interpolating Empirical Orthogonal Functions (DINEOF) is a machine learning method that attempts to reconstruct a spatiotemporal field of observations based on a sparse sample. Data interpolation, or imputation (e.g., predicting the value of the missing observations), is accomplished by leveraging the spatial and temporal correlations present in the sparse sample. The spatial and temporal correlations enable the system to identify K components of variability. As an example, each component comprises:
a set of spatially varying weights, φ(s₁)[k], φ(s₂)[k], . . . , φ(s_{N})[k], where k refers to the k-th component and N denoted the number of unique locations; and
a time series of coefficients, λ₁, λ₂, . . . , λ_{M}, where M represents the number of unique instants, not necessarily equally spaced apart.

According to DINOF, if an observation zₜ(s) is missing in a dataset, the system imputes the missing value/observation based on a function, such as the function described in Equation 17.

zₜ(s)=Σₖ₌₁^{K}ϕ(s)^{[k]}λₜ^{[k]} (17)

Based on the predictions from the previous modules (e.g., the spatial module, the weather module, the diurnal cycle module, the principal component module, etc.), the system first compute the corresponding residuals. The system can compute the residuals according to a function such as the function described in Equation 18.

Yₜ^{*}(s)=Yₜ(s)−( [symbol] (x^{space}(s)))+ [symbol] (xₜ^{weather})+ [symbol] (xₜ^{hour})+ [symbol] (t)) (18)

If a particular location s was sampled n_{d}(s) times on the same day d, then system can compute a daily mean residual according to Equation 19.

$${Z}_{d} ( s ) = \frac{1}{{n}_{d} ( s )} {\sum }_{{∀}_{t} : day ( t ) = d} {Y}_{t}^{*} ( s ) \qquad ( 19 )$$

If no observations were made, then the system deems Z_{d}(s) as missing determines to impute a value for the particular location on day d (or such other time point). The system can use an estimation technique (e.g., algorithm) described below to impute the value.

In some embodiments, the system implements an estimation algorithm to determine imputed values. To impute missing values, the system obtains the values of K, {ϕ(s1)^{[k], . . . , φ(s}_{N})^{[k]}} and {λ₁^{[k]}, . . . , λ_{M}^{[k]}}, for all k=1, . . . , K. The system can obtain the values of K via an iterative optimizer. An example of an iterative optimizer is provided below:
(1) Initialize the algorithm:
   set K=1;
   split the sample into a train and a test set;
   place the training observations (e.g., obtained from the train set) in an N×M matrix Z, in which missing observations (e.g., empty cells) are set to an initial value of zero;
   set the iteration counter i=0; and
   define a stoppage criterion (e.g., ΔRMSEₜₑₛₜ<ε).
(2) Perform a low-rank Singular Value Decomposition (SVD) on matrix Z. The low-rank SVD being performed using the current number of K components: Z≈UDV.
(3) In matrix Z, replace all imputed observations with the estimates provided by the low-rank SVD computed in the previous step.
(4) Compare the test observations with the corresponding estimates (e.g., by computing RMSEₜₑₛₜ). If the algorithm has not converged, then increment the iteration counter (i=i+1) and return to step 2.
(5) If i>0, increment the number of components (K=K+1), reset the iteration counter (i=0) and return to step 2.

Once this optimizer has converged, the system has obtained the final low-rank SVD decomposition \hat{Z}=Û\hat{D}\hat{V}. The system uses the SVD decomposition to compute a few quantities of interest:
The observation error variance parameter, σ², based on the discrepancies between the final imputed values and the test observations. The observation error variance parameter may be computed according to Equation 20.

$${σ}^{2} = \frac{1}{{n}_{test}} \sum {( {Z}_{d} ( s ) - \hat{{Z}_{d}} ( s ) )}^{2} \qquad ( 20 )$$

The temporal evolution error variance, τ², based on the K×M matrix of right singular vectors \hat{V}. The temporal evolution error variance may be computed according to Equation 21.

$${τ}^{2} = \frac{1}{K ( M - 1 )} {\sum }_{k = 1}^{K} {\sum }_{d = 2}^{M} {( \hat{V} ( k , d ) - \hat{V} ( k , d - 1 ) )}^{2} \qquad ( 21 )$$

Spatial parameters φ(s)^{[k]}, for s=s₁, . . . , s_{N} and k=1, . . . , K, stem from the N×K matrix of left singular vectors it and the K×K diagonal matrix of singular values D^{\hat{ }}:

ϕ(s)^{[k]}=U(s,k)·D(k,k) (22)

In some embodiments, the DINEOF enables the system to impute missing observations (e.g., empty cells of a matrix representation of the environmental dataset) when the environmental dataset is sparse. However, DINEOF's performance can degrade as sparsity of the dataset increases. The degradation can get to the point that the algorithm is incapable of determining predictions for days where no samples were collected at any location.

According to various embodiments, the system implements a Kalman Filter (KF) to mitigate this problem of DINEOF. The system implements the KF in accordance with the following:
The observation layer for location s and day d is represented by Equation 23.

Z_{d}(s)˜N[Σₖ₌₁^{K}ϕ(s)^{[k}]λ_{d}^{[k]},σ²] (23)
The process layer is given by a random walk represented by Equation 24.

λ_{d}^{[k]}˜N[λ_{d-1}^{[k]},τ²] (24)
In other words, before assimilating the observations collected on day d, we state that the latent coefficients λ_{d}^{[1]}, . . . , λ_{d}^{[K]} equate to the ones estimated on the previous day, plus some amount of Gaussian white noise with mean 0 and variance τ².
Initial conditions are provided by Equation 25.

λ₁^{[k]}˜N[0,τ²] (25)

According to various embodiments, the system uses the Kalman Filter to estimate the Gaussian posterior mean and variance of the temporal coefficients λ_{d}^{[k]}, for all d=1, . . . , M and k=1, . . . , K, given the available observations. The system estimates the Gaussian posterior mean and variance of the temporal coefficients based on using the forward filtering, backward smoothing equations described in the following: C. K. Carter and R. Kohn, “On Gibbs Sampling for State Space Models”, Biometrika. Vol. 81, No. 3 (August, 1994), pp. 541-553 (hereinafter Carter and Kohn); and Fruhwirth-Schnatter, “Data Augmentation and Dynamic Linear Models”, Journal of Time Series Analysis. Vol. 15, Issue 2 (March 1994), pp. 183-202 (hereinafter Fruhwirth-Schnatter. Both Carter and Kohn and Fruhwirth-Schnatter are hereby incorporated by reference in their entireties for all purposes. From these distributions, the system can sample the missing observations P times, Z_{d}(s)^{[1]}, . . . , Z_{d}(s)^{[P]}, thereby obtaining a probabilistic reconstruction of the response variable, for all locations and times (e.g., for all road segments and days).

In some embodiments, the system generates an augmented environmental dataset, such as a dataset that comprises collected/sampled values or imputed values for all locations and time points during a predefined period of time (e.g., during a length of a contract). For example, the system combines the output of the various modules (e.g., the spatial module, the weather module, the diurnal cycle module, the principal component module, the hyperlocal module, etc.) in REMAQ, to generate the final reconstruction of the environmental dataset (e.g., pollutant concentrations at all road segments and time points (hours) of a contract).
(1) If the system determines that Yt(s) is not missing, then the system uses exp(Yₜ(s)) as the best estimate of the environmental characteristic (e.g., the pollutant concentration) at road segment s, at hour t (e.g., at contract hour t, etc.). The Yt(s) designates a softplus-corrected log-concentration);
(2) If the system determines Yt(s) is missing, then the system uses the sample defined by Equations 26 and 27.

exp( [symbol] (s)+Z_{d}(s)^{[1]}), . . . ,exp( [symbol] (s)+Z_{d}(s)^{[P]}) (26)

where

[symbol]

(s)= [symbol] (x^{space}(s))+ [symbol] (xₜ^{weather})+ [symbol] (xₜ^{hour})+ [symbol] (t) (27)

The system can assess the extent of the uncertainty of the estimate (e.g., imputed value) based on the spread of the sample computed using Equation 26. One advantage of using a sample-based approach is that the system can also construct spatial and temporal aggregations (e.g., census-tract baselines, monthly baselines, etc.) that take spatiotemporal correlations into account, because such correlations are embedded in the DKF algorithm, used to generate the sample.

A useful way to think about this augmented dataset is as if it were a data cube, D, with dimensions P×N×M. The general element of this data cube, D(p, s, t) is equal to a (softplus-corrected) measurement if one was made at time t and location s, or equal to a simulated value if no measurements were made.

The foregoing equations are merely illustrative examples. In various embodiments, the system may implement various equations in connection with processing the environmental data and/or making predictions pertaining to the environmental data.

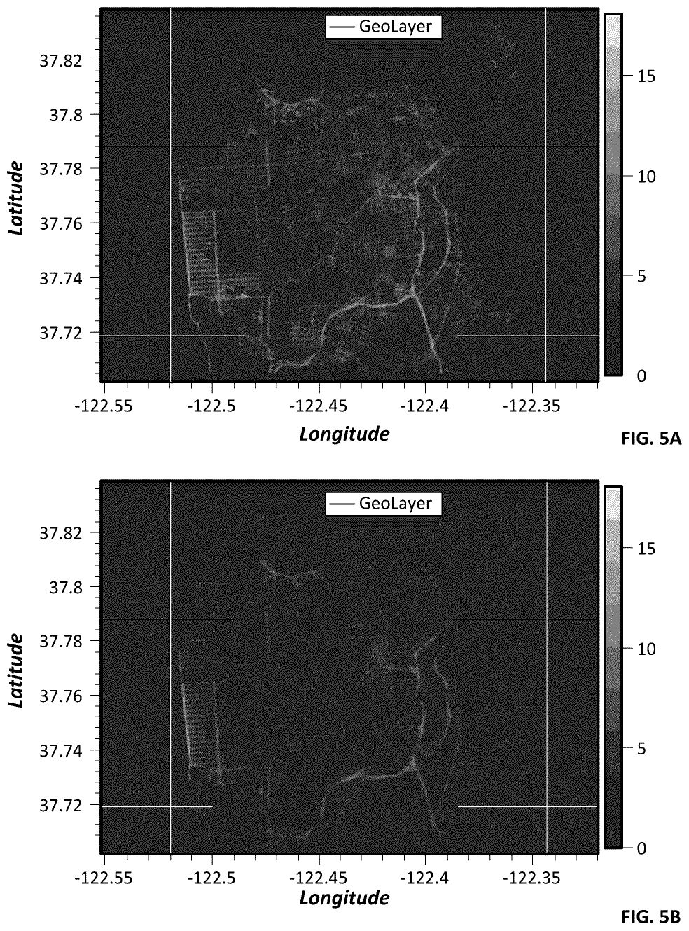

FIG. 5A illustrates an example of an environmental data baseline generated by a raw mean of pass values according to various embodiments. FIG. 5B illustrates an example of an environmental data baseline generated by a spatial module according to various embodiments.

Mobile sampling artifacts, in the shape of polygonal regions that coincide with census tracts, are visible in the raw mean of the pass values (e.g., FIG. 5A), but such sampling artifacts are not as readily apparent in the environmental data baseline (e.g., FIG. 5B). Heavily polluted roads are correctly identified in the raw mean baseline but not in the spatial module baseline. The lack of such correct identification in the spatial module baseline (e.g., FIG. 5B) is a challenge encountered when generating this first version of the baseline: to capture spatially smooth predictions, with salient gradients where possible (such as the coastal highs and the high-altitude lows), while avoiding any mobile sampling artifacts. Subsequent modules (e.g., weather modules, diurnal cycle modules, etc.) will attempt to capture the hyperlocal variability missed by the spatial module.

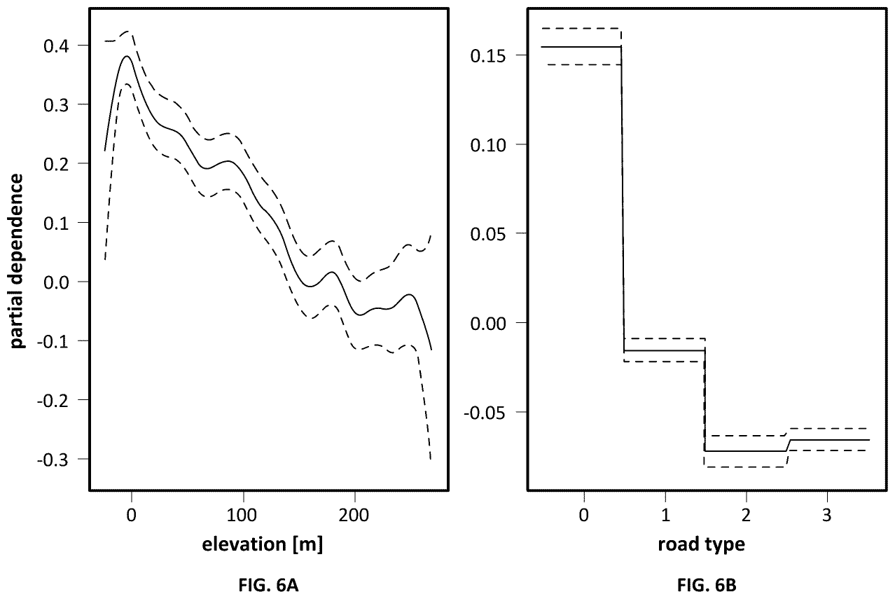

FIG. 6A illustrates an example of a model of the effect of elevation on pollutant concentrations according to various embodiments. FIG. 6B illustrates an example of a model of the effect or a road type on pollutant concentrations according to various embodiments. The dashed lines in FIGS. 6A and 6B respectively correspond to the 95% confidence intervals of the effect estimates.

As shown in FIG. 6A, the model generated according to various embodiments is very flexible as it tries to relate elevation (e.g., the altitude of the road segment) with the pollutant concentration (e.g., the PM2.5 concentration). In the example shown, generally (e.g., on average) the higher the location measured (e.g., sampled, imputed, etc.) the lower the observed pollutant concentration is expected to be. However, as shown in the graph of FIG. 6A, the relationship between elevation and pollutant concentration is non-linear. In some embodiments, because of the uncertainty provided by the GAM, the system does not rule out the possibility that elevation has a monotonically decreasing impact on PM2.5 concentrations.

In the example shown in FIG. 6B, road type 0 corresponds to a highway, road type 1 corresponds to a major road, road type 2 corresponds to other road types, and road type 3 corresponds to a residential road type. As illustrated in FIG. 6B, highways typically have significantly higher PM 2.5 concentrations than other types of road segments

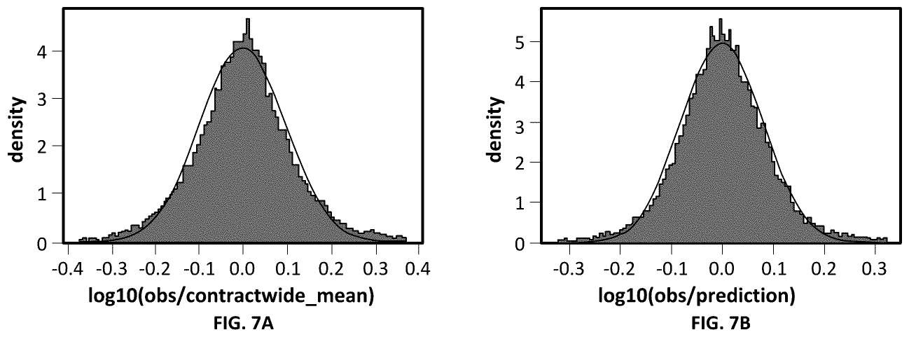

FIGS. 7A and 7B respectively illustrate examples of histograms of null model and spatial module residuals for a particular pollutant according to various embodiments. The histograms of the null model, as defined below, (e.g., FIG. 7A) and the spatial module residuals (e.g., FIG. 7B) are based on N=25970 road segments and pollutant (e.g., PM2.5) concentrations.

A null model is a model with no predictors. For example, the null model simply captures the average of the data. Therefore, any competing model should present smaller residuals, as a sign of improved fit. As illustrated in FIG. 7B, a small amount of shrinkage in the distribution of residuals (R2=4%) is observed. Because almost 95% of log-transformed residuals occur in the interval (−0.2, 0.2), most observations generally fall within 20% of the prediction. In terms of the shape of the distributions of the null model and the spatial module residuals, the residuals look symmetric and roughly gaussian, although both tails look too thick to be precisely gaussian. Because of the shape of the distribution, the system determines that the model could employ additional spatial features, to capture a higher fraction of data variability.

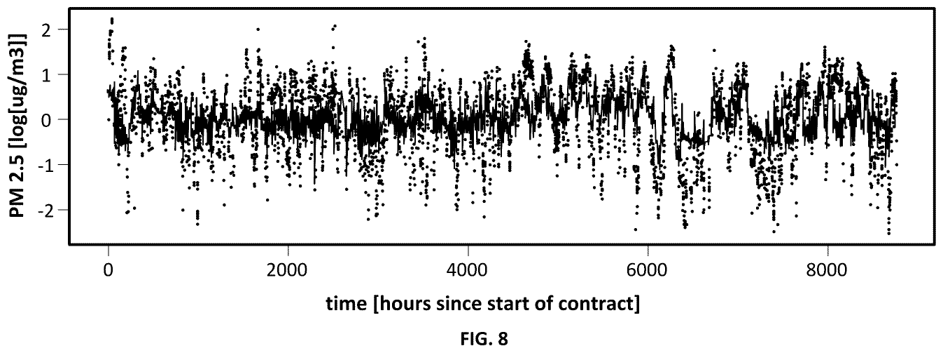

FIG. 8 illustrates an example of temporal dynamics of spatially averaged residuals from a spatial module for a particular pollutant and predictions from a weather module according to various embodiments.

In some embodiments, the weather module is used to assess (e.g., determine) the extent to which weather variables influence the temporal dynamics of spatially averaged residuals from the spatial module. In other words, the system uses the weather module to assess the skill of a weather-based GAM to predict all the variability in the pass data that could not be captured by smooth geographical predictions.

In the example shown, the temporal dynamics of spatially averaged residuals from the PM2.5 spatial module (e.g., the dotted representation) is illustrated with predictions from the weather module (e.g., the solid line representation).

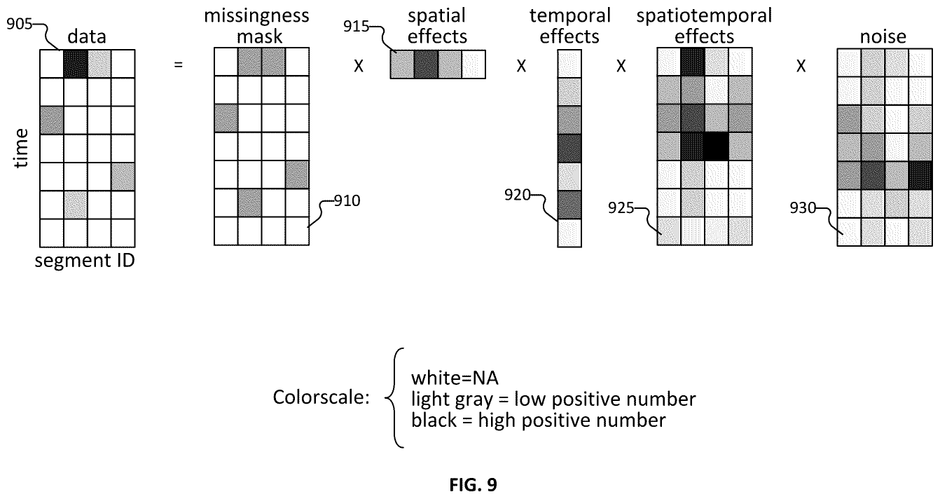

FIG. 9 illustrates a decomposition of an environmental dataset to contributions from the effects of a set of features according to various embodiments. In some embodiments, the system generates an augmented environmental dataset or otherwise determines a model for making predictions at certain locations and times based on the assumption that an observation is based on something happening over space (e.g., an effect having spatial variability), something that happened over time (e.g., the effect having temporal variability) and something that happened over space-time (e.g., the effect having spatiotemporal variability).

In the example shown, environmental dataset 905 can be represented/organized into a matrix having dimensions of time and space (e.g., road segments). The white cells correspond to cells for which no observation was collected. The grey and black cells correspond to cells in which observations were sampled. As illustrated in the missingness mask representation 910 that identifies cells for which observations are collected, the pollutant associated with environmental dataset 905 was not observed everywhere and at only a few times. The system can deconstruct the environmental dataset 905 into (i) effects observed over space (e.g., effects having spatial variability) as represented by the spatial effect component 915, (ii) effects observed over time (e.g., effects having temporal variability) as represented by temporal effect component 920, (iii) effects observed over space-time (e.g., effects having spatiotemporal variability) as represented by spatiotemporal effect component 925, and (iv) effects of noise as represented by noise component 930.

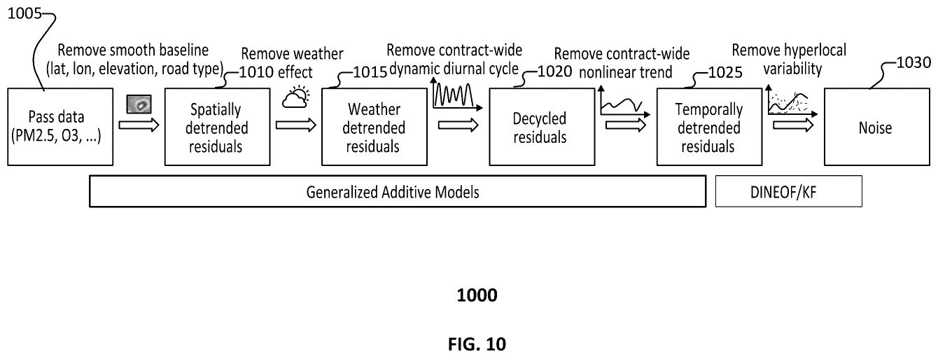

FIG. 10 illustrates a process for analyzing an environmental dataset to determine contributions from the effects of a set of features according to various embodiments. In the example shown, the system implements process 1000 to deconstruct the environmental dataset into a set of components (e.g., features that affect the variability of the environmental data).

The system obtains an environmental dataset 1005 that is based at least in part on collected sample data (e.g., pass data from a mobile sensor platform traveling and sampling along a drive plan during a session). Although not shown in FIG. 10, in some embodiments, the system pre-processes the environmental dataset 1005, such as to resolve negative pollutant concentrations or other data irregularities (e.g., irregularities arising from sensor malfunction or sensor error).

In some embodiments, the system determines the spatial feature component of the environmental dataset 1005. The spatial feature components may comprise topography effects (e.g., the effects of latitude, longitude, or elevation), road characteristics (e.g., road type classification, such as highway, rural, road surface, number of lanes, etc.). Various other effects that cause spatial variability may be identified. The system determines the spatial feature component based on passing the environmental dataset 1005 through a generalized additive model, such as the spatial module described herein. In response to determining the spatial feature component, the system removes the spatial feature component from the environmental dataset 1005 to obtain a spatially detrended residual data 1010.

In some embodiments, the system determines the weather component of the environmental dataset 1005. The system determines the effect of weather on the environmental dataset by passing the spatially detrended residual data 1010 through a generalized additive model such as the weather module described herein. In response to determining the weather component, the system removes the weather component from the spatially detrended residual data 1010 to obtain weather detrended residual data 1015.

In some embodiments, the system determines the dynamic diurnal cycle component of the environmental dataset 1005. The system determines the dynamic diurnal cycle component by passing the weather detrended residual data 1015 through a generalized additive model such as the diurnal cycle module described herein. In response to determining the dynamic diurnal cycle component, the system removes the dynamic diurnal cycle component from the weather detrended residual data 1015 to obtain decycled residual data 1020.

In some embodiments, the system determines the nonlinear trend component of the environmental dataset 1005. The system determines the nonlinear trend component by passing the decycled residual data 1020 through a generalized additive model such as the principal component module described herein. In response to determining the non-linear trend component, the system removes the non-linear trend component from the decycled residual data 1020 to obtain temporally detrended residual data 1025.

In some embodiments, the system determines the noise component of the environmental dataset 1005. For example, the system determines the noise component after the effects of spatial variability, weather variability, temporal variability, and spatiotemporal variability. The system can determine the noise component by passing the temporally detrended residual data 1025 through a DKF to remove hyperlocal variability. In response to determining the hyperlocal variability, the system removes the hyperlocal variability temporally detrended residual data 1025 to obtain the noise component 1030.

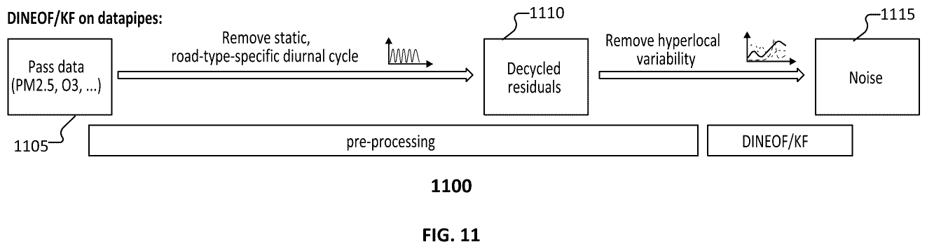

FIG. 11 illustrates a process for using a DINEOF/Kalman Filter (DKF) filter to analyze an environmental dataset to determine contributions from the effects of a set of features according to various embodiments. In the example shown, the system implements process 1100 to deconstruct an environmental dataset 1105 into (i) the effects of spatially-based features and temporally-based features, and (ii) the effect of noise. The system determines the respective effects without use of additional external information (e.g., only information within the external dataset is used). For example, the system uses a road type as a spatially-based feature and diurnal cycles as a temporally-based feature.

As illustrated, the system obtains the environmental dataset 1105. The system can pre-process the environmental dataset 1105 to remove static, road-type-specific diurnal cycles to obtain decycled residual data 1110. In response to obtaining the decycled residual data 1110, the system determines the hyperlocal variability component. In response to determining the hyperlocal variability component, the system removes the hyperlocal variability component from the decycled residual data 1110 to obtain a noise component 1115. In some embodiments, the system determines the noise component by passing the decycled residual data 1110 through a DKF to remove the hyperlocal variability component.

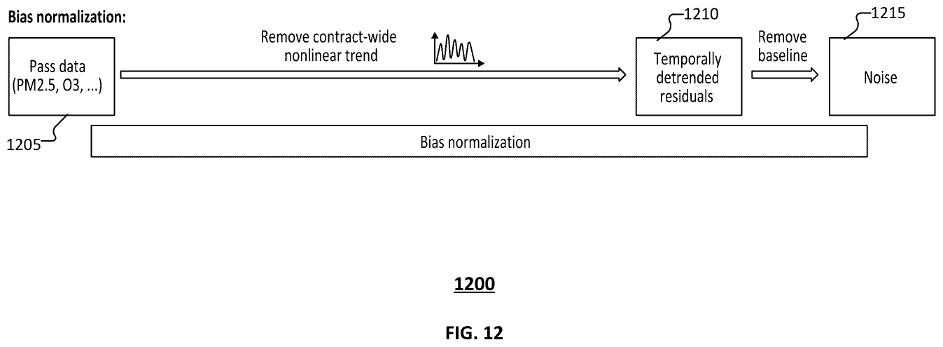

FIG. 12 illustrates a process for using a bias normalization to analyze an environmental dataset to determine contributions from the effects of a set of features according to various embodiments. In the example shown, the system obtains environmental dataset 1205, removes a non-linear trend from over the predefined time period (e.g., a contract length over which environmental data was sampled) to obtain temporally detrended residuals 1210. The system removes a baseline from the temporally detrended residuals 1210 to obtain a noise component 1215.

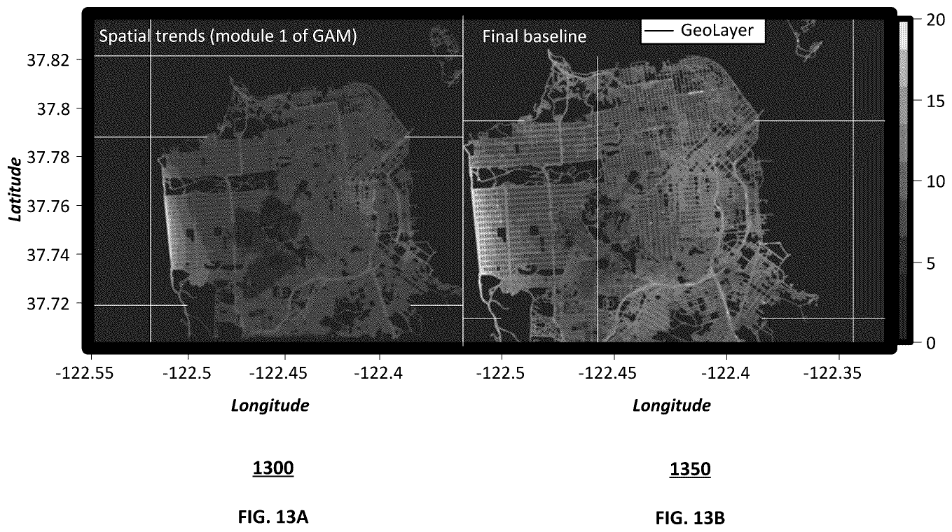

FIG. 13A illustrates a spatial component for a pollutant concentration according to various embodiments. FIG. 13B illustrates a baseline for a pollutant concentration over a geographic region according to various embodiments. FIGS. 13A and 13B illustrate maps 1300 and 1350 of concentrations of PM2.5 over a predefined area. In the example shown, map 1300 illustrates the result from determining the spatial trends, such as by passing the environmental data through a generalized additive model such as the spatial module described herein. In the example shown, the final baseline 1350 has relatively more variability than the spatial trend.

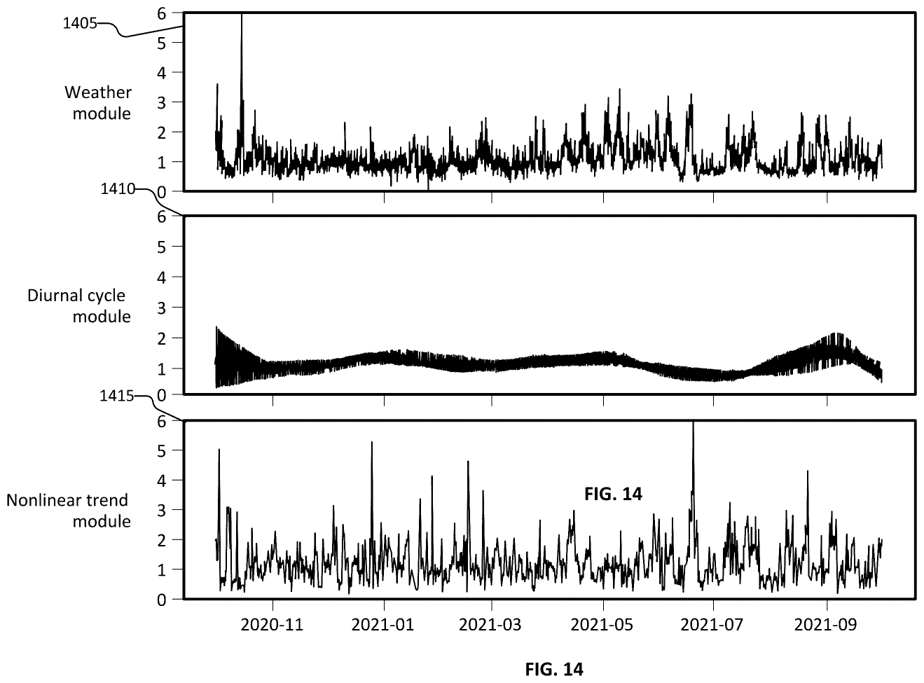

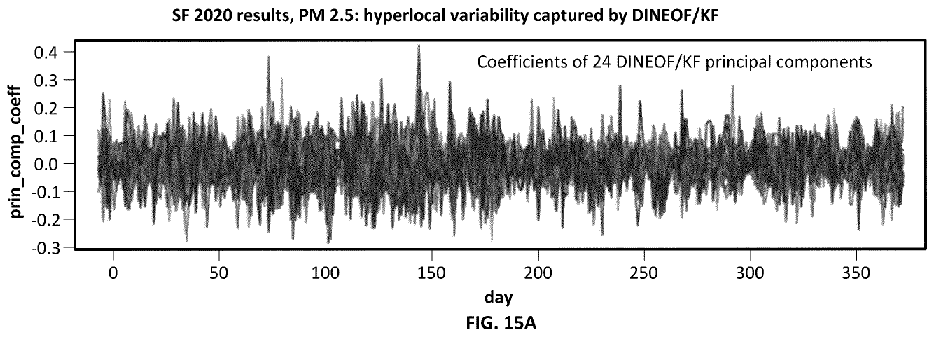

FIG. 14 illustrates the components for weather, diurnal cycle, and nonlinear trends for an environmental dataset according to various embodiments. In the example shown, the system uses the weather module, diurnal cycle module and non-linear trend module (e.g., the principal component module) to extract the various components from the environmental data, such as weather component 1405 corresponding to a weather contribution to the prediction of environmental data, the diurnal cycle component 1410 corresponding to a diurnal cycle contribution to the prediction of environmental data, and the nonlinear trend component 1415. The contributions to the prediction of environmental data are shown on graphs in which the y-axis corresponds to the environmental characteristics (e.g., the pollutant concentration) and the horizontal axis is time.

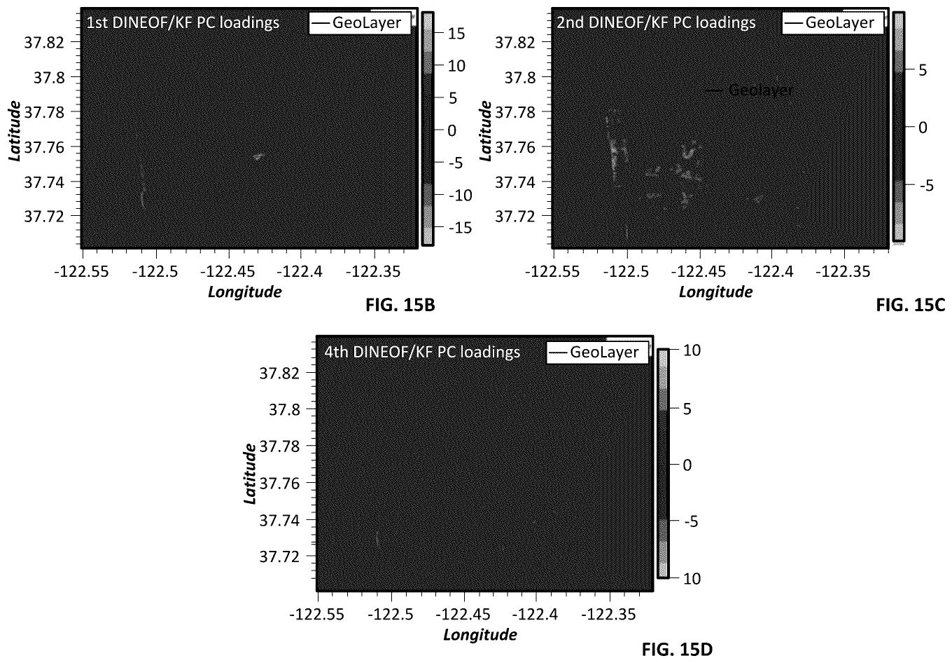

FIG. 15A illustrates a hyperlocal variability component of an environmental dataset for a particular pollutant extracted using a DKF filter. FIGS. 15B-15D illustrate examples of principal component (PC) loadings obtained from an environmental dataset for a particular pollutant extracted using a DKF filter.

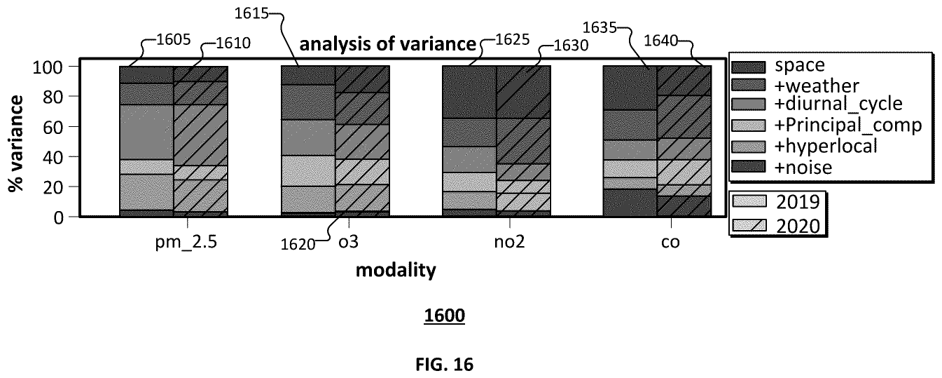

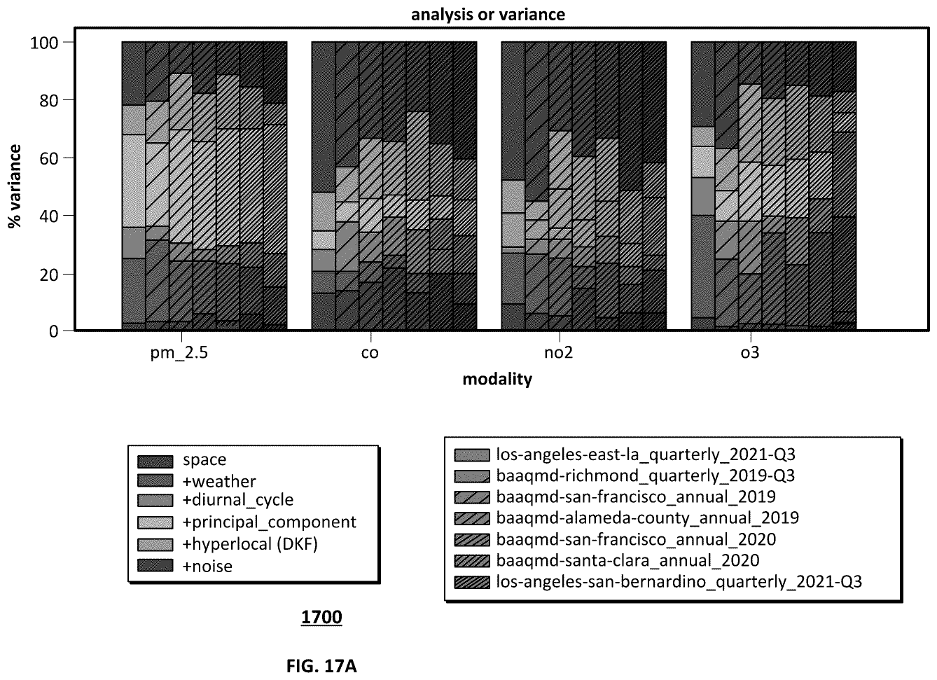

FIG. 16 illustrates an example of the various contributions, from a set of features to an environmental dataset for various pollutants. As illustrated, representation 1600 illustrates an analysis of variance of the different components of features over time. For example, representation 1600 provides an analysis with respect to plurality of pollutants—PM2.5, ozone, nitrogen dioxide, and carbon monoxide. The set of features comprise an effect of a spatially variable feature, an effect of weather feature, an effect of a diurnal cycle feature, an effect of a principal component feature (e.g., a non-linear trend contribution), an effect of a hyperlocal variability feature, and an effect of noise.

Stacked bar chart 1605 illustrates the components of the set of features for environmental data pertaining to PM2.5 concentrations collected over 2019. Conversely, stacked bar chart 1610 illustrates the same components for environmental data collected over 2020. As illustrated, the variance in the PM2.5 concentrations for each feature is relatively constant over the 2019 and 2020 datasets.

Stacked bar chart 1615 illustrates the components of the set of features for environmental data pertaining to ozone concentrations collected over 2019. Conversely, stacked bar chart 1620 illustrates the same components for environmental data collected over 2020. As illustrated, the variance in the ozone concentrations for each feature is relatively constant over the 2019 and 2020 datasets.

Stacked bar chart 1625 illustrates the components of the set of features for environmental data pertaining to nitrogen dioxide concentrations collected over 2019. Conversely, stacked bar chart 1630 illustrates the same components for environmental data collected over 2020. As illustrated, the variance in the nitrogen dioxide concentrations for each feature is relatively constant over the 2019 and 2020 datasets.

Stacked bar chart 1635 illustrates the components of the set of features for environmental data pertaining to carbon monoxide concentrations collected over 2019. Conversely, stacked bar chart 1640 illustrates the same components for environmental data collected over 2020. As illustrated, the variance in the carbon monoxide concentrations for each feature is relatively constant over the 2019 and 2020 datasets.

As indicated by the results depicted in FIG. 16, in various embodiments the system is able to decompose the spatio-temporal variability of pollutant concentrations into signals that are roughly constant over time, as opposed to a spurious decomposition. As a result, the system can attempt to learn about the contribution of different factors to the dynamics of pollution.

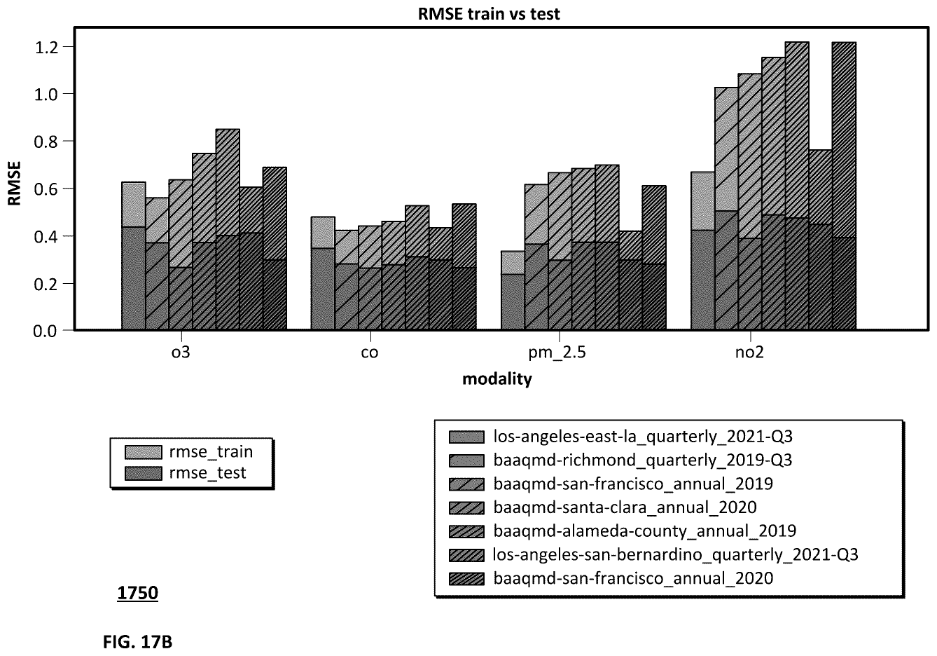

FIG. 17A illustrates an example of a variance for contributions associated with a set of features for various pollutants. As illustrated, representation 1700 illustrates an analysis of variance of the different components of features over contracts. A contract may correspond to a defined geographic region over which environmental data is collected for a predefined period of time (e.g., a calendar quarter, a year, etc.). For example, representation 1600 provides an analysis with respect to plurality of pollutants—PM2.5, ozone, nitrogen dioxide, and carbon monoxide. The change in shading or cross-hatching denotes a different contract. As illustrated in representation 1700, the variance for each feature is relatively constant across the set of contract datasets.

FIG. 17B illustrates an example of the root-mean-square error (RMSE) associated with a model for a set of contracts across various pollutants. As illustrated, representation 1750 illustrates an analysis of a measure of how the model fits the environmental dataset for a set of contracts. For example, representation 1750 illustrates the RMSE for the model determined for a set of pollutants (e.g., ozone, carbon monoxide, PM2.5, and nitrogen dioxide). In the example shown, the RMSE for both the train datasets and the test datasets is relatively constant across contracts.

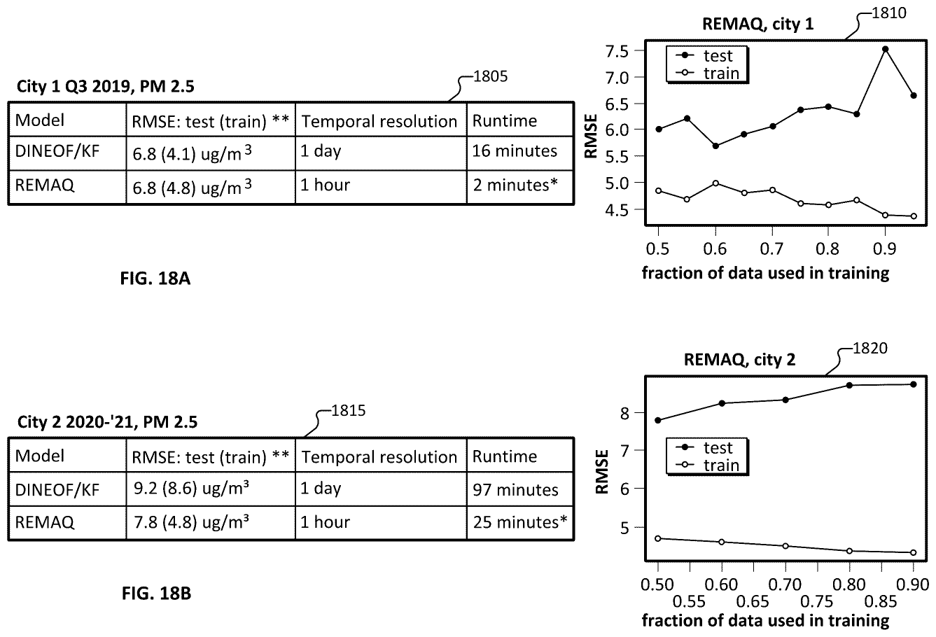

FIG. 18A illustrates a comparison of a DKF model and a residual model for air quality (REMAQ) model generated for an environmental dataset corresponding to a particular pollutant according to various embodiments. In the example shown, chart 1805 corresponding to a particular contract (e.g., monitoring of PM2.5 over a predefined region for a predefined period of time) contrasts a DKF model determined using a DKF such by process 1100 of FIG. 11 versus a REMAQ model determined using the REMAQ technique such as process 1000 of FIG. 10. The REMAQ model provides a model having greater temporal resolution and a lower RMSE (e.g., when using a 50% train to 50% test data split) than the DKF model. Further, the REMAQ model is less computationally expensive (e.g., the runtime to determine the REMAQ model is faster than the determining of the DKF model). For example, use of the DKF model alone requires a very computationally burdensome pre-processing algorithm to remove the diurnal cycle. However, the use of the REMAQ does not require use of this computationally burdensome pre-processing algorithm. The temporal resolution of the REMAQ allows for a very fine environmental dataset, which provides a better representation of environmental characteristics at a particular time. Chart 1810 illustrates the RMSE of the REMAQ model as a function of the percentage of data used in training.

FIG. 18B illustrates a comparison of a DKF model and a residual model for air quality (REMAQ) model generated for an environmental dataset corresponding to a particular pollutant according to various embodiments. In the example shown, chart 1815 corresponding to a particular contract (e.g., monitoring of PM2.5 over a predefined region for a predefined period of time) contrasts a DKF model determined using a DKF such by process 1100 of FIG. 11 versus a REMAQ model determined using the REMAQ technique such as process 1000 of FIG. 10. The REMAQ model provides a model having greater temporal resolution and a lower RMSE (e.g., when using a 50% train to 50% test data split) than the DKF model. Further, the REMAQ model is less computationally expensive (e.g., the runtime to determine the REMAQ model is faster than the determining of the DKF model). The temporal resolution of the REMAQ allows for a very fine environmental dataset, which provides a better representation of environmental characteristics at a particular time. Chart 1820 illustrates the RMSE of the REMAQ model as a function of the percentage of data used in training.

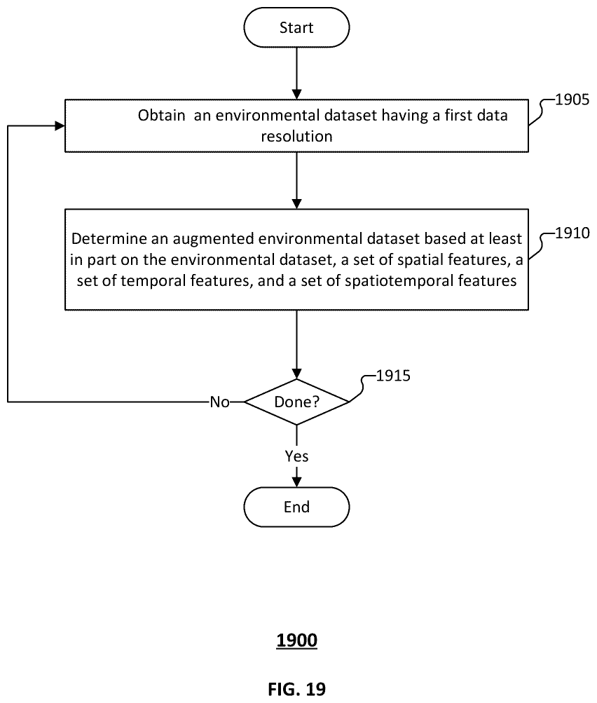

FIG. 19 illustrates a method for augmenting environmental data according to various embodiments. At 1905, the system obtains an environmental dataset having a first data resolution. At 1910, the system determines an augmented environmental dataset based at least in part on the environmental dataset, a set of spatial features, a set of temporal features, and a set of spatiotemporal features. At 1915, a determination is made as to whether process 1900 is complete. In some embodiments, process 1900 is determined to be complete in response to a determination that no further augmented environmental datasets are to be determined, an administrator indicates that process 1900 is to be paused or stopped, etc. In response to a determination that process 1900 is complete, process 1900 ends. In response to a determination that process 1900 is not complete, process 1900 returns to 1905.

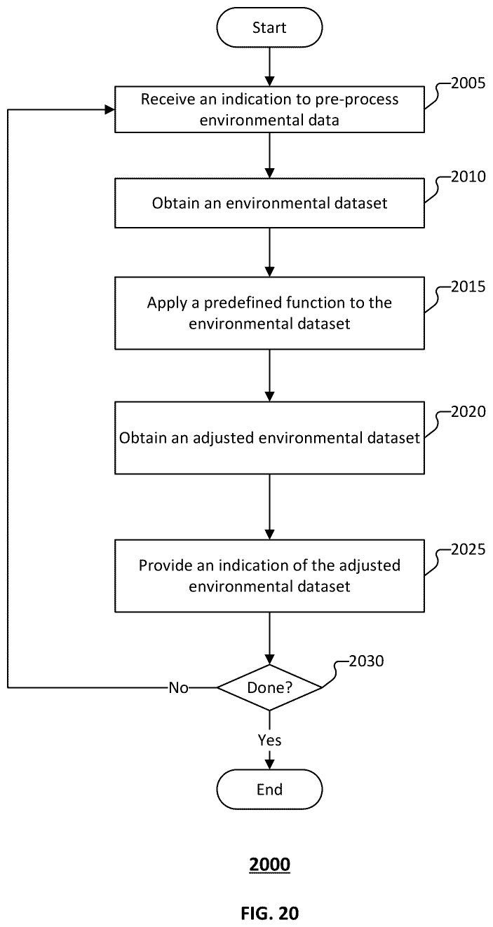

FIG. 20 illustrates a method for pre-processing environmental data according to various embodiments. At 2005, the system receives an indication to pre-process environmental data. At 2010, the system obtains an environmental dataset. At 2015, the system applies a predefined function for the environmental dataset. For example, the system applies the predefined function in connection with pre-processing the environmental dataset, such as to resolve negative concentration observations or other sensor errors. At 2020, the system obtains an adjusted environmental dataset. At 2025, the system provides an indication of the adjusted environmental dataset. At 2030, a determination is made as to whether process 2000 is complete. In some embodiments, process 2000 is determined to be complete in response to a determination that pre-processing an environmental dataset is complete, no further environmental datasets are to be complete, an administrator indicates that process 2000 is to be paused or stopped, etc. In response to a determination that process 2000 is complete, process 2000 ends. In response to a determination that process 2000 is not complete, process 2000 returns to 2005.

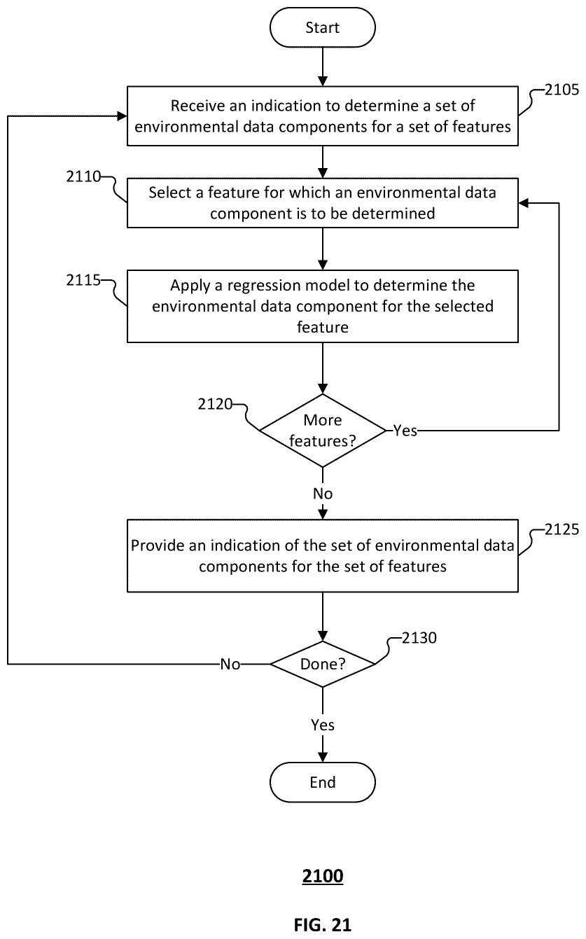

FIG. 21 illustrates a method for determining the components for a set of features that are contributed to a prediction of environmental data according to various embodiments. At 2105, the system receives an indication to determine a set of environmental data components for a set of features. At 2110, the system selects a feature for which an environmental data component is to be determined. Examples of types of features in the set of features include a spatial feature, a temporal feature, a spatiotemporal feature, a weather feature, etc. Various other types of features may be implemented. At 2115, the system applies a regression model to determine the environmental data component for the selected feature. The regression model may be a generalized additive model. At 2120, the system determines whether components for more features are to be determined. In response to determining that components for more features are to be determined, process 2100 returns to 2110 and process 2100 iterates over 2110 to 2120 until no further components for more features are to be determined. At 2125, the system provides an indication of the set of environmental components for the set of features. At 2130, a determination is made as to whether process 2100 is complete. In some embodiments, process 2100 is determined to be complete in response to a determination no further contributions to the prediction of environmental data by a set of features are to be determined, no further environmental datasets are to be complete, an administrator indicates that process 2100 is to be paused or stopped, etc. In response to a determination that process 2100 is complete, process 2100 ends. In response to a determination that process 2100 is not complete, process 2100 returns to 2105.

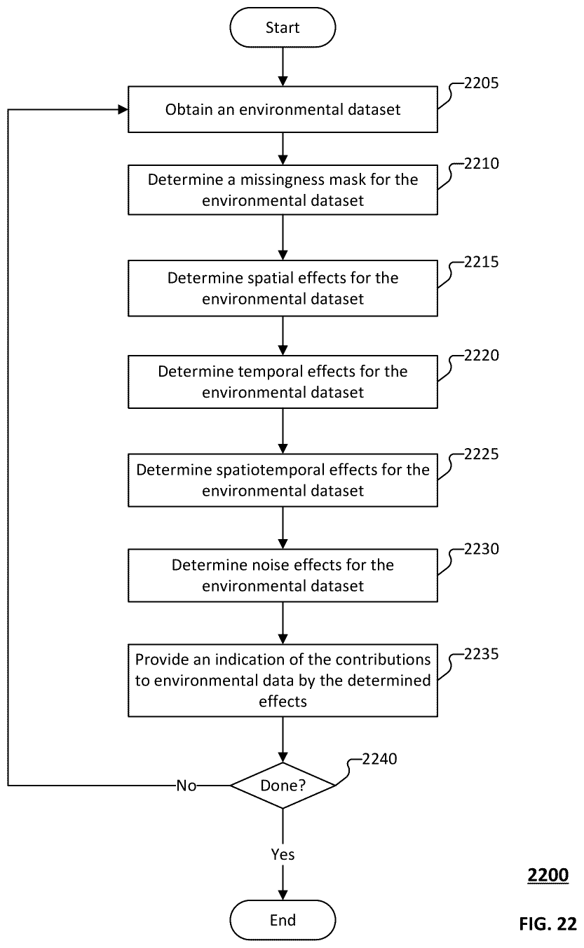

FIG. 22 illustrates a method for determining the components for a set of features that are contributed to a prediction of environmental data according to various embodiments. At 2205, the system obtains an environmental dataset. At 2210, the system determines a missingness mask for the environmental dataset. For example, the system determines (location, time) tuples for which observations were collected and represented in the environmental dataset and (location, time) tuples for which no observations were collected. At 2215, the system determines spatial effects for the environmental dataset. At 2220, the system determines temporal effects for the environmental dataset. At 2225, the system determines spatiotemporal effects for the environmental dataset. At 2230, the system determines noise effects for the environmental dataset. At 2235, the system provides an indication of the contributions to the prediction of environmental data by the determined effects. At 2240, a determination is made as to whether process 2200 is complete. In some embodiments, process 2200 is determined to be complete in response to a determination no further contributions to the prediction of environmental data by a set of features are to be determined, no further environmental datasets are to be complete, an administrator indicates that process 2200 is to be paused or stopped, etc. In response to a determination that process 2200 is complete, process 2200 ends. In response to a determination that process 2200 is not complete, process 2200 returns to 2205.

According to various embodiments, various orders of steps 2215-2230 may be implemented.

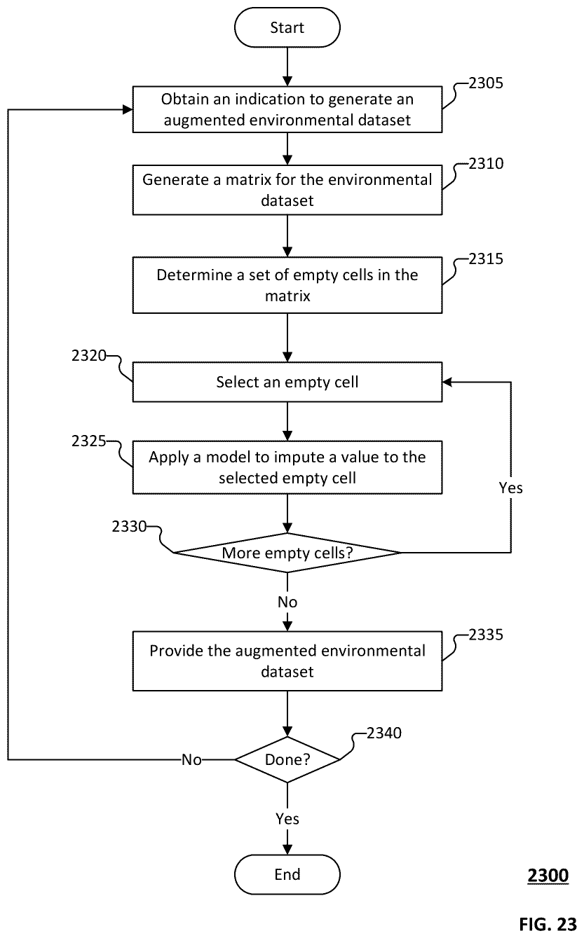

FIG. 23 illustrates a method for determining an augmented environmental dataset according to various embodiments. In some embodiments, the system implements process 2300 to reducing the number of missing or null values in an environmental dataset. For example, the system obtains a sparse environmental dataset (e.g., raw pass data) and determines an environmental dataset (e.g., the augmented environmental dataset) having no missing values.

At 2305, the system obtains an indication to that an augmented environmental dataset is to be generated. At 2310, the system generates a matrix for the environmental dataset. At 2315, the system determines a set of empty cells in the matrix. At 2320, the system selects and empty cell. At 2325, the system applies a model to impute a value to the selected empty cell. At 2330, the system determines whether a value is to be imputed for another empty cell. In response to determining that the value is to be imputed for another empty cell, process 2300 returns to 2320 and process 2300 iterates over 2320-2330 until no further values are to be imputed. Conversely, in response to determining that no further values are to be imputed for an empty cell(s), process 2300 proceeds to 2335. At 2335, the system provides the augmented environmental dataset. At 2340, a determination is made as to whether process 2300 is complete. In some embodiments, process 2300 is determined to be complete in response to a determination no further environmental datasets are to be analyzed, no further augmented environmental datasets are to be generated, no further environmental datasets are to be complete, an administrator indicates that process 2300 is to be paused or stopped, etc. In response to a determination that process 2300 is complete, process 2300 ends. In response to a determination that process 2300 is not complete, process 2300 returns to 2305.

Various examples of embodiments described herein are described in connection with flow diagrams. Although the examples may include certain steps performed in a particular order, according to various embodiments, various steps may be performed in various orders and/or various steps may be combined into a single step or in parallel.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method for augmenting environmental data, comprising:
obtaining, by one or more processors, an environmental dataset, the environmental dataset comprising a sparse environmental dataset; and
determining an augmented environmental dataset based at least in part on the environmental dataset, a set of spatial features, a set of temporal features, and a set of spatiotemporal features;
wherein the augmented environmental dataset has less missing or null values than a the environmental dataset.

2. The method of claim 1, further comprising:
predicting, based on the model, an environmental characteristic at a predefined location and at a predefined time.

3. The method of claim 2, wherein the environmental characteristic comprises a pollutant concentration.

4. The method of claim 1, wherein the determining the augmented environmental dataset includes:
generating a matrix for the environmental dataset, wherein the matrix comprises a plurality of cells that respectively correspond to a particular location and a particular time;
determining a set of empty cells in the matrix for which the environmental dataset has no observed value; and
for each of the set of empty cells, determine an imputed value for the environmental data, the imputed value being determined based at least in part on the set of spatial features, the set of temporal features, and the set of spatiotemporal features.

5. The method of claim 1, wherein:
the environmental data includes pass data collected from set of mobile sensors that is mounted to a set of vehicles; and
the set of vehicles are directed to drive a predefined drive plan within a particular geographic area.

6. The method of claim 1, wherein the determining the augmented environmental dataset comprises:
determining a noise component for the environmental dataset, the noise component being determined based at least in part on one or more of (i) removing a spatial variability component, (ii) removing a weather component, (iii) removing a temporal variability component, and (iv) removing a hyperlocal variability component.

7. The method of claim 6, wherein the noise component is used in connection with determining imputed values for the augmented environmental dataset.

8. The method of claim 6, wherein one or more of the spatial variability component, the temporal variability component, and the weather component is determined based at least in part on a corresponding generalized additive model.

9. The method of claim 8, wherein the generalized additive model is a regression model.

10. The method of claim 6, wherein the spatial variability component is determined based at least in part on a generalized additive model for a set of spatial features.

11. The method of claim 6, wherein the temporal variability component is determined based at least in part on a generalized additive model for a set of temporal features.

12. The method of claim 6, wherein the noise component is determined based at least in part on:
removing the spatial variability component to obtain a spatially detrended residual data;
removing the weather component from the detrended residual data to obtain weather detrended residual data;
removing a dynamic diurnal cycle component from the detrended residual data to obtain decycled residual data;
removing a non-linear trend component from the decycled residual data to obtain temporally detrended residual data; and
removing a hyperlocal variability component from the temporally detrended residual data to obtain the noise component.

13. The method of claim 12, wherein a Data Interpolating Empirical Orthogonal Functions (DINEOF)/Kalman Filter (DKF) filter is used to remove the hyperlocal variability component from temporally detrended residual data.

14. The method of claim 6, wherein the temporal variability component comprises a dynamic diurnal cycle component and a nonlinear trend component.

15. The method of claim 1, wherein the set of spatial features is determined based at least in part on one or more of a longitude, a latitude, an altitude, and a road segment type corresponding a particular location in a predefined geographic map for which an environmental data value is determined.

16. The method of claim 1, wherein the environmental dataset is pre-processed before the augmented environmental dataset is determined.

17. The method of claim 1, wherein pre-processing the environmental dataset includes adjusting the environmental dataset based at least in part on negative concentration observations in the environmental dataset.

18. The method of claim 17, wherein adjusting the environmental dataset comprises shifting negative concentration observations in the environmental dataset without shifting a mode of the environmental dataset.

19. A system for augmenting environmental data, comprising:
a processor configured to:
obtain an environmental dataset, the environmental dataset comprising a sparse environmental dataset; and
determine an augmented environmental dataset based at least in part on the environmental dataset, a set of spatial features, a set of temporal features, and a set of spatiotemporal features;
wherein the augmented environmental dataset has less missing or null values than a the environmental dataset; and
a memory coupled to the processor and configured to provide the processor with instructions.

20. A computer program product for sensing air quality with a sensor platform, the computer program product being embodied in a tangible computer readable storage medium and comprising computer instructions for:
obtaining, by one or more processors, an environmental dataset, the environmental dataset comprising a sparse environmental dataset; and
determining an augmented environmental dataset based at least in part on the environmental dataset, a set of spatial features, a set of temporal features, and a set of spatiotemporal features;
wherein the augmented environmental dataset has less missing or null values than a the environmental dataset.
